SEQUENCE LISTING (10 sequences)

SEQ ID NO: 1 - protein, 1132 aa, Mus musculus
MGMACLTMTEMEATSTSPVHQNGDIPGSANSVKQIEPVLQVYLYHSLGQAEGEYLKFPSGEYVAEEICVAASKACGITPVYHNMFALMSETERIWYPPNHVFHIDESTRHDILYRIRFYFPHWYCSGSSRTYRYGVSRGAEAPLLDDFVMSYLFAQWRHDFVHGWIKVPVTHETQEECLGMAVLDMMRIAKEKDQTPLAVYNSVSYKTFLPKCVRAKIQDYHILTRKRIRYRFRRFIQQFSQCKATARNLKLKYLINLETLQSAFYTEQFEVKESARGPSGEEIFATIIITGNGGIQWSRGKHKESETLTEQDVQLYCDFPDIIDVSIKQANQECSNESRIVTVHKQDGKVLEIELSSLKEALSFVSLIDGYYRLTADAHHYLCKEVAPPAVLENIHSNCHGPISMDFAISKLKKAGNQTGLYVLRCSPKDFNKYFLTFAVERENVIEYKHCLITKNENGEYNLSGTKRNFSNLKDLLNCYQMETVRSDSIIFQFTKCCPPKPKDKSNLLVFRTNGISDVQISPTLQRHNNVNQMVFHKIRNEDLIFNESLGQGTFTKIFKGVRREVGDYGQLHKTEVLLKVLDKAHRNYSESFFEAASMMSQLSHKHLVLNYGVCVCGEENILVQEFVKFGSLDTYLKKNKNSINILWKLGVAKQLAWAMHFLEEKSLIHGNVCAKNILLIREEDRRTGNPPFIKLSDPGISITVLPKDILQERIPWVPPECIENPKNLNLATDKWSFGTTLWEICSGGDKPLSALDSQRKLQFYEDKHQLPAPKWTELANLINNCMDYEPDFRPAFRAVIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLRLIMEYLPYGSLRDYLQKHKERIDHKKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPVEFMRMIGNDKQGQMIVFHLIELLKSNGRLPRPEGCPDEIYVIMTECWNNNVSQRPSFRDLSLRVDQIRDSIAA

SEQ ID NO: 2 - DNA, 5030 bp, Mus musculus
gggcccccgctctgggcgagctcgcgcggcgtcggcgcctcggccgcggccccggctccggaggcgcccgtccgtcccggcgagggctgcaggcggctgccgggtccgccgctgcgggatgtgagtgcgggccaagcccgcgcgggagggcccccgagggcccagcctggcggccgctcgaccctgcgcccggcccgggggctccctagacctcggcccgcccgacgcgtctcggaggagcggatcctgcgcgcctcggggcgctcctgtgtggcccgacccgtgtcgccggttcctgaagcaggcgacgggaacaagatgtgaactgttttccctccccagaagaagaggccctttttttccctcccgcgaaggccaatgttctgaaaaaagctctagatgggaatggcctgccttacaatgacagaaatggaggcaacctccacatctcctgtacatcagaatggtgatattcctggaagtgctaattctgtgaagcagatagagccagtccttcaagtgtatctgtaccattctcttgggcaagctgaaggagagtatctgaagtttccaagtggagagtatgttgcagaagaaatttgtgtggctgcttctaaagcttgtggtattacgcctgtgtatcataatatgtttgcgttaatgagtgaaaccgaaaggatctggtacccacccaatcatgtcttccacatagacgagtcaaccaggcatgacatactctacaggataaggttctacttccctcattggtactgtagtggcagcagcagaacctacagatacggagtgtcccgtggggctgaagctcctctgcttgatgactttgtcatgtcttacctttttgctcagtggcggcatgattttgttcacggatggataaaagtacctgtgactcatgaaactcaggaagagtgtcttgggatggcggtgttagacatgatgagaatagctaaggagaaagaccagactccactggctgtctataactctgtcagctacaagacattcttaccaaagtgcgttcgagcgaagatccaagactatcacattttaacccggaagcgaatcaggtacagatttcgcagattcattcagcaattcagtcaatgtaaagccactgccaggaacctaaaacttaagtatcttataaacctggaaaccctgcagtctgccttctacacagaacagtttgaagtaaaagaatctgcaagaggtccttcaggtgaggagatttttgcaaccattataataactggaaacggtggaattcagtggtcaagagggaaacataaggaaagtgagacactgacagaacaggacgtacagttatattgtgatttccctgatattattgatgtcagtattaagcaagcaaaccaggaatgctcaaatgaaagtagaattgtaactgtccataaacaagatggtaaagttttggagatagaacttagctcattaaaagaagccttgtcattcgtgtcattaattgacgggtattacagactaactgcggatgcgcaccattacctctgcaaagaggtggctcccccagctgtgctcgagaacatacacagcaactgccacggcccaatatcaatggattttgccattagcaaactaaagaaggcgggtaaccagactggactatatgtgctacgatgcagccctaaggacttcaacaaatactttctgacctttgctgttgagcgagaaaatgtcattgaatataaacactgtttgattacgaagaatgagaatggagaatacaacctcagcgggactaagaggaacttcagtaaccttaaggaccttttgaattgctaccagatggaaactgtgcgctcagacagtatcatcttccagtttaccaaatgctgccccccaaagccaaaagataaatcaaaccttctcgtcttcagaacaaatggtatttctgatgttcagatctcaccaacattacagaggcataataatgtgaatcaaatggtgtttcacaaaatcaggaatgaagatttaatatttaatgaaagtcttggccaaggtacttttacaaaaatttttaaaggtgtaagaagagaagttggagattatggtcaactgcacaaaacggaagttcttttgaaagtcctagataaagcacataggaactattcagagtctttcttcgaagcagcaagcatgatgagtcagctttctcacaagcatttggttttgaattatggtgtctgtgtctgtggagaggagaacattctggttcaagaatttgtaaaatttggatcactggatacatacctgaagaagaacaaaaattccataaatatattatggaaacttggagtggctaagcagttggcatgggccatgcattttctagaagaaaaatcccttattcatgggaatgtgtgtgctaaaaatatcctgcttatcagagaagaagacaggagaacggggaacccacctttcatcaaacttagtgatcctggcattagcattacagttctaccgaaggacattcttcaggagagaataccatgggtacctcctgaatgcattgagaatcctaaaaatctcaatctggcaacagacaagtggagcttcgggaccactctgtgggagatctgcagtggaggagataagcccctgagtgctctggattctcaaagaaagctgcagttctatgaagataagcatcagcttcctgcacccaagtggacagagttagcaaaccttataaataattgcatggactatgagccagatttcaggcctgctttcagagctgtcatccgtgatcttaacagcctgtttactccagattatgaactactaacagaaaatgacatgctaccaaacatgagaataggtgccctagggttttctggtgcttttgaagacagggaccctacacagtttgaagagagacacttgaagtttctacagcagcttggcaaaggtaacttcgggagtgtggagatgtgccgctatgacccgctgcaggacaacactggcgaggtggtcgctgtgaagaaactccagcacagcactgaagagcacctccgagactttgagagggagatcgagatcctgaaatccttgcagcatgacaacatcgtcaagtacaagggagtgtgctacagtgcgggtcggcgcaacctaagattaattatggaatatttaccatatggaagtttacgagactatctccaaaaacataaagaacggatagatcacaaaaaacttcttcaatacacatctcagatatgcaagggcatggaatatcttggtacaaaaaggtatatccacagggacctggcaacaaggaacatattggtggaaaatgagaacagggttaaaataggagacttcggattaaccaaagtcttgccgcaggacaaagaatactacaaagtaaaggagccaggggaaagccccatattctggtacgcacctgaatccttgacggagagcaagttttctgtggcctcagatgtgtggagctttggagtggttctatacgaacttttcacatacatcgagaagagtaaaagtccacccgtggaatttatgcgaatgattggcaatgataaacaagggcaaatgattgtgttccatttgatagagctactgaagagcaacggaagattgccaaggccagaaggatgcccagatgagatttatgtgatcatgacagagtgctggaacaacaatgtgagccagcgtccctccttcagggacctttcccttcgggtggatcaaatccgggacagtatagctgcgtgaaagagatggccttcactcagagaccaagcagacttccagaaccagaacaaagctctgtagccttgtgtctacacatccttatcatgatgctagctaggcagaagaaactgtgacgccgtctgctcaaagctttggaaacgccgtgcgtttgtttctacaccatctgtaaaaaccactgctcaagtctggcagcatgcttgtgggctgatgcatggagctcaccacagagtctctgcatctcctctggcagaagaagaaaatagacagttttcaactcagcttttttgagaaatggaaaaaattataatgtaaattttgcagtgtaggaaatacacagaacatacatgtacagtttttaccacgtggagtgtataatactttggcctcttgtgtgatttacatgagggctgatgtttgttaatgttttctaatttttccataggtgatctataataacttcatgatacaaattaaaatgctcagaaaattaaataccttttctttcttgctcatctttgctgctctgttagggcctgtagttccatgccactgtaaatatttttcaaagcaaaaggaacaaaggtctagttttacttgtataggaaatcttcctgaccctaaagaattttgaaatgggacaatcttacaaagacacagcacaatctattttcttatttgttttccttgtgtttatggtaaatgtgtttttaaatagaactgtctccaactttttctatgactataatgaatataatgaatccttttataattttgagattaagaatgccagaaatacttccctcccttgaactgttaactgccaatgagatgtttcaatccctaagacttgtcattatacattaaattgaagcataagccatacataatttgtaaaatgtacaagctctttaagatgctttctgggttggaacatggccaaagactgtgtgagaggcctccaggtttgccattacagctgtgagagaaacgtcctttcctcagcgtctcccagacagatgggaagggaagggtgtggggcacatgctgttcatcccattcaagaataccagcaggcgtgtgacgcacatctcgagatgagaagagagagcgtacatcttttcaaagtataaataattgttatcatccttcgtgctgctccctcttagacaatgtcatgtcaaaaataaaacaagatttttgtgtgttatttatacaagattttaaatatttgg

SEQ ID NO: 3 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 4 - DNA, 5097 bp, Homo sapiens
ctgcaggaaggagagaggaagaggagcagaagggggcagcagcggacgccgctaacggcctccctcggcgctgacaggctgggccggcgcccggctcgcttgggtgttcgcgtcgccacttcggcttctcggccggtcgggcccctcggcccgggcttgcggcgcgcgtcggggctgagggctgctgcggcgcagggagaggcctggtcctcgctgccgagggatgtgagtgggagctgagcccacactggagggcccccgagggcccagcctggaggtcgttcagagccgtgcccgccccggggcttcgcagaccttgacccgccgggtaggagccgcccctgcgggctcgagggcgcgctctggtcgcccgatctgtgtagccggtttcagaagcaggcaacaggaacaagatgtgaactgtttctcttctgcagaaaaagaggctcttcctcctcctcccgcgacggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctagggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccaccagtaaaagacattaatgagaattccttagcaaggattttgtaagaagtttcttaaacattgtctgttaacatcactcttgtctggcaaaagaaaaaaaatagactttttcaactcagctttttgagacctgaaaaaattattatgtaaattttgcaatgttaaagatgcacagaatatgtatgtatagtttttaccacagtggatgtataataccttggcatcttgtgtgatgttttacacacatgagggctggtgttcattaatactgttttctaatttttccatagttaatctataattaattacttcactatacaaacaaattaagatgttcagataattgaataagtacctttgtgtccttgttcatttatatcgctggccagcattataagcaggtgtatacttttagcttgtagttccatgtactgtaaatatttttcacataaagggaacaaatgtctagttttatttgtataggaaatttccctgaccctaaataatacattttgaaatgaaacaagcttacaaagatataatctattttattatggtttcccttgtatctatttgtggtgaatgtgttttttaaatggaactatctccaaatttttctaagactactatgaacagttttcttttaaaattttgagattaagaatgccaggaatattgtcatcctttgagctgctgactgccaataacattcttcgatctctgggatttatgctcatgaactaaatttaagcttaagccataaaatagattagattgttttttaaaaatggatagctcattaagaagtgcagcaggttaagaattttttcctaaagactgtatatttgaggggtttcagaattttgcattgcagtcatagaagagatttatttcctttttagaggggaaatgaggtaaataagtaaaaaagtatgcttgttaattttattcaagaatgccagtagaaaattcataacgtgtatctttaagaaaaatgagcatacatcttaaatcttttcaatta

SEQ ID NO: 5 - protein, 235 aa, Mus musculus
MSTESMIRDVELAEEALPQKMGGFQNSRRCLCLSLFSFLLVAGATTLFCLLNFGVIGPQRDEKFPNGLPLISSMAQTLTLRSSSQNSSDKPVAHVVANHQVEEQLEWLSQRANALLANGMDLKDNQLVVPADGLYLVYSQVLFKGQGCPDYVLLTHTVSRFAISYQEKVNLLSAVKSPCPKDTPEGAELKPWYEPIYLGGVFQLEKGDQLSAEVNLPKYLDFAESGQVYFGVIAL

SEQ ID NO: 6 - DNA, 1619 bp, Mus musculus
cctcagcgaggacagcaagggactagccaggagggagaacagaaactccagaacatcttggaaatagctcccagaaaagcaagcagccaaccaggcaggttctgtccctttcactcactggcccaaggcgccacatctccctccagaaaagacaccatgagcacagaaagcatgatccgcgacgtggaactggcagaagaggcactcccccaaaagatggggggcttccagaactccaggcggtgcctatgtctcagcctcttctcattcctgcttgtggcaggggccaccacgctcttctgtctactgaacttcggggtgatcggtccccaaagggatgagaagttcccaaatggcctccctctcatcagttctatggcccagaccctcacactcagatcatcttctcaaaattcgagtgacaagcctgtagcccacgtcgtagcaaaccaccaagtggaggagcagctggagtggctgagccagcgcgccaacgccctcctggccaacggcatggatctcaaagacaaccaactagtggtgccagccgatgggttgtaccttgtctactcccaggttctcttcaagggacaaggctgccccgactacgtgctcctcacccacaccgtcagccgatttgctatctcataccaggagaaagtcaacctcctctctgccgtcaagagcccctgccccaaggacacccctgagggggctgagctcaaaccctggtatgagcccatatacctgggaggagtcttccagctggagaagggggaccaactcagcgctgaggtcaatctgcccaagtacttagactttgcggagtccgggcaggtctactttggagtcattgctctgtgaagggaatgggtgttcatccattctctacccagcccccactctgacccctttactctgacccctttattgtctactcctcagagcccccagtctgtatccttctaacttagaaaggggattatggctcagggtccaactctgtgctcagagctttcaacaactactcagaaacacaagatgctgggacagtgacctggactgtgggcctctcatgcaccaccatcaaggactcaaatgggctttccgaattcactggagcctcgaatgtccattcctgagttctgcaaagggagagtggtcaggttgcctctgtctcagaatgaggctggataagatctcaggccttcctaccttcagacctttccagattcttccctgaggtgcaatgcacagccttcctcacagagccagcccccctctatttatatttgcacttattatttattatttatttattatttatttatttgcttatgaatgtatttatttggaaggccggggtgtcctggaggacccagtgtgggaagctgtcttcagacagacatgttttctgtgaaaacggagctgagctgtccccacctggcctctctaccttgttgcctcctcttttgcttatgtttaaaacaaaatatttatctaacccaattgtcttaataacgctgatttggtgaccaggctgtcgctacatcactgaacctctgctccccacgggagccgtgactgtaatcgccctacgggtcattgagagaaataaa

SEQ ID NO: 7 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 8 - DNA, 1669 bp, Homo sapiens
ctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaa

SEQ ID NO: 9 - DNA, 22 bp, Artificial Sequence
  Synthetic oligonucleotide primer
gactacggtcaactgcatgaaa

SEQ ID NO: 10 - DNA, 20 bp, Artificial Sequence
  Synthetic oligonucleotide primer.
ccatgccaactgtttagcaa

(19) United States
(12) Patent Application Publication
(10) Pub. No.: US 2010/0310563 A1
(43) Pub. Date: Dec. 9, 2010

(54) METHODS FOR TREATING INDUCED CELLULAR PROLIFERATIVE DISORDERS

(76) Inventors: Thomas G.P. Bumm, Portland, OR (US); Michael W.N. Deininger, Portland, OR (US)

Correspondence Address: Klarquist Sparkman LLP, 121 SW Salmon Street, Suite 1600, Portland, OR, 97204, US

(21) Appl. No.: 12/745,540

(22) PCT Filed: Nov. 28, 2008
(86) PCT No.: PCT/US08/85073

§ 371 Date: May 28, 2010

Related U.S. Application Data
(60) Provisional application No. 60/991,666, filed on Nov. 30, 2007.

(51) Int. Cl. A61K 39/395; A61K 31/7052; A61K 31/7076; A61K 31/551; A61K 31/5377; A61K 31/4439; A61K 31/44; A61K 31/277; A61K 31/519; C12Q 1/68; C12Q 1/02; A61P 35/00; A61P 35/02

(52) U.S. Cl. 424/134.1; 514/44 A; 424/158.1; 424/142.1; 424/133.1; 424/145.1; 514/45; 514/211.08; 514/235.8; 514/341; 514/357; 514/521; 514/265.1; 435/6; 435/29

(57) ABSTRACT

Methods are provided for treating a subject with a cellular proliferative disorder that include administering to the subject a therapeutically effective amount of a JAK2 inhibitor and a therapeutically effective amount of a TNF-alpha inhibitor. In addition, methods are provided herein for determining if a subject with a cellular proliferative disorder would benefit from treatment with an agent that inhibits tumor necrosis factor (TNF)-alpha. Methods are also provided for identifying an agent of use in treating a subject with a cellular proliferative disorder or with a predisposition for cellular proliferative disorder. The methods include contacting an isolated cell expressing an activating mutation in the JAK2 protein with a test agent, and detecting the amount of tumor necrosis factor (TNF)-alpha produced by the cell.

### PRIORITY CLAIM

The application claims the benefit of U.S. Provisional Application No. 60/991,666, filed Nov. 30, 2007, which is incorporated by reference herein in its entirety.

### STATEMENT OF GOVERNMENT SUPPORT

This invention was made with United States Government support under grant HL082978-01, awarded by the National Institutes of Health. The Government has certain rights in the invention.

### FIELD

This application relates to the field of cellular proliferative disorders, such as myeloproliferative disorders and specifically to the treatment of cellular proliferative disorders and methods for the identification of agents of use for treating these disorders.

### BACKGROUND

The myeloproliferative disorders (MPD) are a heterogeneous group of disorders characterized by expansion of one or more of the myeloid lineages. Classically, MPDs have included chronic myelogenous leukemia (CML), polycythemia vera (PV), essential thrombocythemia (ET), and idiopathic myelofibrosis (IMF). More recently, the definition has been broadened to include a variety of additional entities, frequently referred to as “atypical MPD,” among them are chronic myelomonocytic leukemia (CMML), chronic neutrophilic leukemia (CNL) and chronic eosinophilic leukemia (CEL).

Constitutively active tyrosine kinases are thought to play a central role in the pathogenesis of MPD. Examples include BCR-ABL in CML and the FIP1L 1-platelet-derived growth factor-a fusion protein in the hypereosinophilic syndrome.

More recently, a mutation (V617F) in the pseudokinase (JH2) domain of the Janus-Activated Kinase-2 (JAK 2) kinase has been identified in 65% to 97% of patients with PV, 35% to 95% of patients with IMF, and 23% to 43% of patients with ET. JAK2V617F has also been observed at lower frequencies in patients with CMML, CNL, systemic mastocytosis, and other rare MPDs. Based on structural considerations, it is believed that the V617F mutation disrupts the autoinhibitory function of the JH2 domain, leading to constitutive activation of the kinase. The constitutive activation of JAK2 is believed to be the causative agent in MPDs in which the JAK2 activating mutant have been observed. Therefore, there is a need for therapy for the treatment of JAK2 induced in MPD.

### SUMMARY

Mutations in the Janus-Activated Kinase-2 (JAK 2) kinase have been implicated as the causative agent in many different MPDs. Thus, the need exists for new treatments for these disorders. This disclosure meets these needs.

Disclosed herein is the surprising discovery that, cellular proliferative disorders caused by JAK2 activating mutations are dependent on the presence of the hormone Tumor Necrosis Factor (TNF)-alpha to grow and cause the MPD. This discovery was based on the observation that mice with normal TNF-alpha production and an activating JAK2 mutation developed MPS very quickly, while mice that lack TNF-alpha do not develop MPD.

In several embodiments, methods of treating a subject with a cellular proliferative disorder are provided herein. The methods include administering to the subject a therapeutically effective amount of an inhibitor of JAK2 kinase activity and a therapeutically effective amount of an inhibitor TNF-alpha activity, thereby treating the cellular proliferative disorder. Pharmaceutical compositions are also provided herein. The compositions include a therapeutically effective amount of an inhibitor of JAK2 kinase activity and a therapeutically effective amount of an inhibitor TNF-alpha activity.

In further embodiments, methods are provided herein for determining if a subject with a cellular proliferative disorder would benefit from treatment with an agent that inhibits the activity of TNF-alpha. The methods include detecting the presence of an activating mutation in JAK2 in a biological sample from the subject. The presence of the activating mutation in JAK2 indicates that the subject would benefit from treatment with the agent that inhibits TNF-alpha activity.

In several embodiments, methods are provided for identifying an agent of use in treating a subject with a cellular proliferative disorder, such as a myeloproliferative disorder and/or cancer, or with a predisposition for cellular proliferative disorder. The method includes contacting an isolated cell expressing an activating mutation in the JAK2 protein with a test agent, and detecting the amount of TNF-alpha produced by the cell. The amount of TNF-alpha produced by the cell is compared to a control. A reduction in the amount of TNF-alpha produced by the cell relative to the control indicates that the agent as useful for the treatment of a subject with the cellular proliferative disorder or with a predisposition for developing the cellular proliferative disorder.

The foregoing and other objects, features, and advantages of the invention will become more apparent from the following detailed description, which proceeds with reference to the accompanying figures.

### BRIEF DESCRIPTION OF THE DRAWINGS

FIGS. 1A-1C is a set of graphs and digital images of Western blots showing that JAK2V617F induces cytokine hypersensitivity in BaF/3 cells. FIG. 1A is a graph showing total number of viable cells as measured by 3-(4-5-dimethylthiazol-2-yl)-5-(3-carboxymethoxyphenyl)-2-(4-sulfophenyl)-2H-tetrazolium salt (MTS) assay. Cells stably expressing JAK2V617F (black solid line, triangles), JAK2WT (gray dashed line, squares), and parental BaF/3 cells control (gray solid line, circles). Points, mean of triplicate results; bars, SD. FIG. 1B is a graph showing viability as determined by propidium iodide exclusion. Cells stably expressing JAK2V617F (black solid line, triangles), JAK2WT (gray dashed line, squares), and parental BaF/3 cells control (gray solid line, circles). Points, mean of triplicate results; bars, SD. FIG. 1C is a set of Western blots of parental BaF/3 and BaF/3-expressing JAK2V617F cells.

FIGS. 2A-2B is a set of graphs and digital images of mice and peripheral blood smears showing that JAK2V617F induces erythrocytosis and leukocytosis in BALB/c mice. FIG. 2A is a set of graphs showing peripheral blood counts of empty vector (circles), JAK2WT (squares), and JAK2V617F (triangles) mice over 80-day time period. Points, mean of triplicate results; bars, SD. Top, left, hematocrit; top, right, WBC; bottom, left, platelets; bottom, right, skin plethora in a JAK2V617F mouse but not JAK2WT mouse. FIG. 2B is a set of digital images of peripheral blood smears of an empty vector, JAK2WT, and JAK2V617F mouse. Top, H&E (HE); bottom, reticulin stain (RE). Original magnifications, x400 and x630.

FIGS. 3A-3D are digital images of histopathological sections of mouse tissues. Representative histologic sections from spleen (FIG. 3A), liver (FIG. 3B), and bone marrow (BM; FIG. 3C and FIG. 3D) from an empty vector, JAK2WT, and JAK2V617F mouse, respectively. H&E and reticulin fiber stain. Original magnifications, x100, x200, x630, and x-1,000.

FIGS. 4A-4B are digital images of representative histogram from FACS analysis showing JAK2V617F-induced constitutive JAK2 phosphorylation in peripheral granulocytes. FIG. 4A is a set of histograms showing cells cultured without cytokines of a JAK2V617F mouse (top) and normal control BALB/c mice (bottom). Phosphorylated JAK2 was analyzed in GFP-positive cells versus GFP-negative cells. FIG. 4B is a set of histograms showing GFP-positive cells of a JAK2V617F mouse cultured without cytokines and then stimulated or not for 60 minutes with IL-3 and G-CSF or stimulated in the presence of 10 Amol/L AG-490 (bottom).

FIGS. 5A-5D is a set of bar graphs showing the results of FACS analysis of mouse tissue from mice transplanted with marrow infected with empty vector (white), JAK2WT (gray), and JAK2V617F (black). Columns, mean; bars, SD. FIG. 5A, left, shows Lin−, CD117+ hematopoietic stem cell/progenitor population in bone marrow and spleen of mice; right, ratio JAK2V617F mice versus MSCV empty vector mice of Lin-CD117+ hematopoietic stem cell/progenitor cells, methycellulose colonies without cytokines (−Cytokines), colonies with cytokines plus Epo (+Cytok. +Epo), and colonies with cytokines but no Epo of cells extracted from spleen (+Cytok. −Epo). FIG. 5B, left, shows Lin−, CD117+, Fcγ receptor+/−, and CD34+/−progenitor populations in bone marrow and spleen of mice; right, Lin−, CD117+, CD9+, receptor low, and CD41+ megakaryocyte progenitor population in bone marrow and spleen of mice. FIG. 5C, left, shows Lin−, CD117+, and CD71+/− erythroid progenitors in bone marrow and spleen of mice; right, percentage of EpoR+ cells of Lin+, CD71high/low, and Ter-119high/low erythroblast cells. FIG. 5D shows serum Epo, G-CSF, and TNF-alpha levels of JAK2WT-infected mice (gray) and JAK2V617F-infected mice (black).

FIG. 6 is a table referred to as Table 1 in the text.

FIG. 7 is as graph showing hematocrit levels of transplanted mice over 61 days, starting at day −3 before transplantation. Arrows mark the time of collection of blood plasma samples of V617F positive and WT mice for cytokine analysis. ▴ are JAK2-V617F positive mice, are JAK2-WT positive mice and ▾ are MSCV—empty vector control mice. The gray area is normal range of hematocrit.

FIG. 8 is a graph showing plasma cytokine levels of mice transplanted with JAK2-WT or JAK2-V617F mutant bone marrow, at day 61 of transplantation.

FIG. 9 is a graph showing hematocrit levels of mice over 52 days after bone marrow transplant. TNF-alpha wild type bone marrow carrying MSCV-empty vector, JAK2-WT or JAK2-V617F construct, transplanted into TNF-alpha wild type recipient mice.

FIG. 10 is a graph of hematocrit levels of mice over 52 days after bone marrow transplant. TNF-alpha knock out bone marrow carrying MSCV-empty vector, JAK2-WT or JAK2-V617F construct, transplanted into TNF-alpha wild type recipient mice.

FIG. 11 is a graph of hematocrit levels of mice over 90 days after bone marrow transplant. TNF-alpha knock out bone marrow carrying JAK2-V617F construct, transplanted into TNF-alpha knock out recipient mice.

FIG. 12 is a bar graph showing spleen weight of TNF-alpha wild type mice. Transplanted with TNF-alpha wild type or knock out bone marrow carrying MSCV-empty vector, JAK2-WT or JAK2-V617F construct.

FIG. 13 is a digital image of bone marrow (HE stain) of mice transplanted with JAK2-V617F positive TNF-alpha wild type cells.

FIG. 14 is a digital image of reticulin fiber stain of bone marrow of mice transplanted with JAK2-V617F positive TNF-alpha wild type cells.

FIG. 15 is a digital image of bone marrow (HE stain) of mice transplanted with JAK2-V617F positive TNF-alpha knock out cells.

FIG. 16 is a digital image of a reticulin fiber stain of bone marrow of mice transplanted with JAK2-V617F positive TNF-alpha knock out cells.

FIG. 17 is a digital image of spleen (HE stain) of mice transplanted with JAK2-V617F positive TNF-alpha wild type cells.

FIG. 18 is a digital image of spleen (HE stain) of mice transplanted with JAK2-V617F positive TNF-alpha knock out cells.

FIGS. 19-27 are supporting data.

### SEQUENCE LISTING

The nucleic and amino acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NO: 1 is an exemplary amino acid sequence of mouse JAK2.

SEQ ID NO: 2 is an exemplary nucleic acid sequence of mouse JAK2.

SEQ ID NO: 3 is an exemplary amino acid sequence of human JAK2.

SEQ ID NO: 4 is an exemplary nucleic acid sequence of human JAK2.

SEQ ID NO: 5 is an exemplary amino acid sequence of mouse TNF-alpha.

SEQ ID NO: 6 is an exemplary nucleic acid sequence of mouse TNF-alpha.

SEQ ID NO: 7 is an exemplary amino acid sequence of human TNF-alpha.

SEQ ID NO: 8 is an exemplary nucleic acid sequence of human TNF-alpha.

SEQ ID NO: 9 and 10 are exemplary nucleic acid primers.

### DETAILED DESCRIPTION

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

### I. Terms

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. For example, the term “an inhibitor” includes single or plural inhibitors and can be considered equivalent to the phrase “at least one inhibitor.”

As used herein, the term “comprises” means “includes.” Thus, “comprising an inhibitor” means “including an inhibitor” without excluding other elements. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting.

To facilitate review of the various embodiments of the invention, the following explanations of terms are provided:

Amplification: To increase the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample, for example the number of an JAK2 nucleic acid, such as a mutant JAK2 nucleic acid, for example an JAK2 nucleic acid in which encodes a JAK2 protein with a phenylalanine at position 617 of SEQ ID NO:1 or SEQ ID NO:3 or fragment of SEQ ID NO:1 or SEQ ID NO:3. An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR(RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

Animal: A living multicellular vertebrate organism, a category that includes, for example, mammals and birds. A “mammal” includes both human and non-human mammals, such as mice. “Subject” includes both human and animal subjects, such as mice.

Antibody: A polypeptide ligand including at least a light chain or heavy chain immunoglobulin variable region which specifically binds an epitope of an antigen, such as an epitope of TNF-alpha. The term “specifically binds” refers to, with respect to an antigen, the preferential association of an antibody or other ligand, in whole or part, with this polypeptide, such as TNF-alpha. Examples of antibodies that specifically bind TNF-alpha are known in the art. A specific binding agent, such as an antibody binds substantially only to a defined target. It is recognized that a minor degree of non-specific interaction may occur between a molecule, such as a specific binding agent, and a non-target polypeptide. Nevertheless, specific binding can be distinguished as mediated through specific recognition of the antigen. Although selectively reactive antibodies bind antigen, they can do so with low affinity. Specific binding typically results in greater than 2-fold, such as greater than 5-fold, greater than 10-fold, or greater than 100-fold increase in amount of bound antibody or other ligand (per unit time) to a polypeptide, as compared to a non-target polypeptide.

A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Antibodies can be composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”), that specifically bind the antigen. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Antisense, Sense, and Antigene: Double-stranded DNA (dsDNA) has two strands, a 5′->3′ strand, referred to as the plus strand, and a 3′->5′ strand (the reverse complement), referred to as the minus strand. Because RNA polymerase adds nucleic acids in a 5′->3′ direction, the minus strand of the DNA serves as the template for the RNA during transcription. Thus, the RNA formed will have a sequence complementary to the minus strand and identical to the plus strand (except that U is substituted for T).

Antisense molecules are molecules that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a dsDNA target.

In some embodiments an antisense molecule is designed to target (for example to repress the expression) TNF-alpha, for example using the nucleic acid sequences of TNF-alpha set forth in the accompanying sequence listing. In some embodiments an antisense molecule is designed to target (for example to repress the expression) JAK2, for example using the nucleic acid sequences of JAK2 set forth in the accompanying sequence listing.

Binding or stable binding (of an oligonucleotide): An oligonucleotide binds or stably binds to a target nucleic acid if a sufficient amount of the oligonucleotide forms base pairs or is hybridized to its target nucleic acid, to permit detection of that binding, for example the binding of an oligonucleotide to the nucleic acid sequence of JAK2, or a mutation in the sequence of JAK2, such as a mutation the results in the substitution of a phenylalanine for valine at position 617 of the amino acid sequence of human JAK2.

Binding can be detected by either physical or functional properties of the target:oligonucleotide complex. Binding between a target and an oligonucleotide can be detected by any procedure known to one skilled in the art, including both functional and physical binding assays.

Physical methods of detecting the binding of complementary strands of DNA or RNA are well known in the art, and include such methods as DNase I or chemical footprinting, gel shift and affinity cleavage assays, Northern blotting, dot blotting and light absorption detection procedures. For example, one method that is widely used, because it is so simple and reliable, involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target disassociate from each other, or melt.

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

cDNA (complementary DNA): A piece of DNA lacking internal, non-coding segments (introns) and transcriptional regulatory sequences. cDNA may also contain untranslated regions (UTRs) that are responsible for translational control in the corresponding RNA molecule. cDNA is usually synthesized in the laboratory by reverse transcription from messenger RNA (mRNA) extracted from cells, for example mRNA encoding a JAK2 protein or a mutation thereof, such as the JAK2 V617F mutation.

Cellular proliferative disorder: A disease or condition characterized by increased proliferation of cells. In some examples, a “cellular proliferative disorder” is a cancer, such as a solid tumor or a leukemia. In some examples a “cellular proliferative disorder” is myeloproliferative disorder. In some examples, a cellular proliferative disorder results from an activating mutation in JAK2 kinase, such as the JAK2 V617F mutation. In some examples, a cellular proliferative disorder is AML, such as AML FAB M7.

Chemotherapy: In cancer treatment, chemotherapy refers to the administration of one or more agents (chemotherapeutic agents) to kill or slow the reproduction of rapidly multiplying cells, such as tumor or cancer cells. In a particular example, chemotherapy refers to the administration of one or more agents to significantly reduce the number of tumor cells in the subject, such as by at least about 50%. “Chemotherapeutic agents” include any chemical agent with therapeutic usefulness in the treatment of cancer. Chemotherapeutic agents include kinase inhibitors, such as inhibitors of the tyrosine kinase JAK2. Chemotherapeutic agents also include inhibitors of TNF-alpha activity.

Examples of chemotherapeutic agents can be found for example in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). A chemotherapeutic agent of use in a subject, such as a JAK2 kinase inhibitor, or a TNF-alpha inhibitor can decrease a sign or a symptom of a cancer, or can reduce, stop or reverse the progression, metastasis and/or growth of a cancer, and/or can reduce tumor mass.

Complementarity and percentage complementarity: Molecules with complementary nucleic acids form a stable duplex or triplex when the strands bind, (hybridize), to each other by forming Watson-Crick, Hoogsteen or reverse Hoogsteen base pairs. Stable binding occurs when an oligonucleotide remains detectably bound to a target nucleic acid sequence under the required conditions.

Complementarity is the degree to which bases in one nucleic acid strand base pair with the bases in a second nucleic acid strand. Complementarity is conveniently described by percentage, such as the proportion of nucleotides that form base pairs between two strands or within a specific region or domain of two strands. For example, if 10 nucleotides of a 15-nucleotide oligonucleotide form base pairs with a targeted region of a DNA molecule, that oligonucleotide is said to have 66.67% complementarity to the region of DNA targeted.

A thorough treatment of the qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), Molecular Cloning: A Laboratory Manual, 2nd ed., vol. 1-3, Cold Spring Harbor Laboratory Press, Cold Spring Harbor, N.Y., 1989.

Contacting: Placement in direct physical association including both in solid or liquid form. Contacting can occur in vivo, for example by administering an agent to a subject. “Administration” is the introduction of a composition, such as a composition containing one or more of an inhibitor of TNF-alpha activity and an inhibitor of JAK2 kinase activity, into a subject by a chosen route. For example, if the chosen route is intravenous, the composition is administered by introducing the composition into a vein of the subject. “Administrating” to a subject includes topical, parenteral, oral, intravenous, intra-muscular, sub-cutaneous, inhalational, nasal, or intra-articular administration, among others.

Control: A reference standard. A control can be a standard value a control cell not contacted with an agent. A difference between a test sample and a control can be an increase or conversely a decrease. The difference can be a qualitative difference or a quantitative difference, for example a statistically significant difference. In some examples, a difference is a decrease, relative to a control, of at least about 10%, such as at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater then 500%.

DNA (deoxyribonucleic acid): DNA is a long chain polymer which comprises the genetic material of most living organisms (some viruses have genes comprising ribonucleic acid (RNA)). The repeating units in DNA polymers are four different nucleotides, each of which comprises one of the four bases, adenine, guanine, cytosine and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes JAK2 or TNF-alpha, or a fragment thereof, encompasses both the sense strand and its reverse complement. Thus, for instance, it is appropriate to generate probes or primers from the reverse complement sequence of the disclosed nucleic acid molecules.

Degenerate variant: A polynucleotide encoding a protein of interest that includes a sequence that is degenerate as a result of the genetic code. For example, a polynucleotide encoding JAK2 or TNF-alpha includes a sequence that is degenerate as a result of the genetic code. There are 20 natural amino acids, most of which are specified by more than one codon. Therefore, all degenerate nucleotide sequences are included as long as the amino acid sequence of the JAK2 polypeptide encoded by the nucleotide sequence is unchanged. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Such nucleic acid variations are “silent variations,” which are one species of conservative variations. Each nucleic acid sequence herein that encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Detect: To determine if an agent (such as a signal, particular nucleotide, for example a nucleotide in a JAK2 nucleic acid, or a protein such as TNF-alpha) is present or absent or the level of the agent that is present in a sample. In some examples, this can further include quantification, for example quantification of TNF-alpha in a sample.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence. For example, an oligonucleotide can be complementary to a JAK2 encoding mRNA, a JAK2 encoding DNA, or a JAK2-encoding dsDNA.

“Specifically hybridizable” and “specifically complementary” are terms that indicate a sufficient degree of complementarity such that stable and specific binding occurs between the oligonucleotide (or it's analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na' concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11.

The following is an exemplary set of hybridization conditions and is not limiting:

Very High Stringency (detects sequences that share at least 90% identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (detects sequences that share at least 80% identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (detects sequences that share at least 50% identity)

Hybridization: 6×SSC at RT to 55° C. for 16-20 hours

Wash at least twice: 2×-3×SSC at RT to 55° C. for 20-30 minutes each.

Isolated: An “isolated” biological component (such as a nucleic acid molecule, protein or organelle) has been substantially separated or purified away from other biological components in the cell of the organism in which the component naturally occurs, i.e., other chromosomal and extra-chromosomal DNA and RNA, proteins and/organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids, such as probes and primers.

Inhibiting or treating a disease: Inhibiting the full development of a disease or condition, for example, in a subject who is at risk for a disease such cancer, or a myeloproliferative disease. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other clinical or physiological parameters associated with a particular disease. A “prophylactic” treatment is a treatment administered to a subject who does not exhibit signs of a disease or exhibits only early signs for the purpose of decreasing the risk of developing pathology.

Inhibit: To reduce to a measurable extent. For example to reduce enzymatic activity. In some examples, the kinase activity of a JAK2 kinase is inhibited. In some examples, the activity of TNF-alpha, such as the ability of TNF-alpha to bind its receptor, is inhibited.

Janus Activated Kinase (JAK)/Signal Transducer and Activator of Transcription (STAT): JAKs are cytoplasmic tyrosine kinases that are either constitutively associated with cytokine receptors or recruited to receptors after ligand binding. Stimulation with the ligand results in the catalytic activation of receptor-associated JAKs. This activation results in the phosphorylation of cellular substrates, including the JAK-associated cytokine receptor chains. Some of these phosphorylated tyrosines can serve as coding sites for STAT proteins, which bind to the phosphotyrosines by their SRC-homology 2 (SH2) domains. STAT proteins are also phosphorylated on conserved tyrosine residues, resulting in their dimerization and acquisition of high-affinity DNA-binding activity, which facilitates their action as nuclear transcription factors.

JAK2 kinase acts as an intermediary between membrane-bound cytokine receptors such as the erythropoietin receptor (EpoR), and down-stream members of the signal transduction pathway such as STAT5 (Signal Transducers and Activators of Transcription protein 5). JAK2 is activated when cytokine receptor/ligand complexes phosphorylate the associated JAK2 kinase. JAK2 can then phosphorylate and activate its substrate molecule, for example STAT5, which enters the nucleus and interacts with other regulatory proteins to affect transcription.

The JAK/STAT pathway is one of the most rapid cytoplasmic to nuclear signaling mechanisms. There are a total of four JAK (JAK1-3 and tyrosine kinase 2) and seven STAT proteins (STAT1-4, STAT5A, STAT5b and STAT6). JAKs are relatively large cytoplasmic kinases of about 1,100 amino acids in length, and range in size from about 116 kDa to about 140 kDa. The STAT proteins can dimerize, translocate to the nucleus, and bind DNA. Binding of the STAT proteins to the DNA can result in the activation of transcription (for review see Leonard, Nature Reviews 1: 200-208, 2001).

“STAT inhibitor,” “JAK inhibitor,” and “JAK/STAT inhibitor” are used to refer to any agent capable of down-regulating or otherwise decreasing or suppressing the amount and/or activity of JAK-STAT interactions. JAK inhibitors down-regulate the quantity or activity of JAK molecules. STAT inhibitors down-regulate the quantity or activity of STAT molecules Inhibition of these cellular components can be achieved by a variety of mechanisms known in the art, including, but not limited to binding directly to JAK (for example, a JAK-inhibitor compound binding complex, or substrate mimetic), binding directly to STAT, or inhibiting the expression of the gene, which encodes the cellular components. JAK/STAT inhibitors are disclosed in U.S. Patent Publication No. 2004/0209799).

JAK2 sequences are publicly available. For example, GENBANK® Accession number NM_{—}008413 discloses a mouse JAK2 gene sequence, and GENBANK® Accession numbers NP_{—}032439 disclose a mouse JAK2 protein sequence. In an other examples, GENBANK® Accession number NM_{—}004972 discloses a human JAK2 gene sequence, and GENBANK® Accession number NP_{—}004963 disclose a human JAK2 protein sequence. One skilled in the art will appreciate that JAK2 nucleic acid and protein molecules can vary from those publicly available, such as those having one or more substitutions, deletions, insertions, or combinations thereof, while still retaining JAK2 biological activity, such as kinase activity.

A “JAK2 inhibitor” inhibits the signaling of a JAK2 protein, for example by inhibiting the kinase activity of JAK2. Exemplary JAK2 inhibitors are antibodies that specifically bind JAK2, siRNAs, ribozymes, antisense molecules, and small molecule kinase inhibitors, such as, such as Lestaurtinb (CEP701, CEPHALON®), TG101348 (TargeGen, Inc.), CYT387 (Cytopia), AZ960 (AstraZeneca), SGI-1252 (SUPERGEN®), WP1066, AG490 (A.G. Scientific Inc.), INCB18424 (Incyte), and SB1518 (S*BIO™).

Kinase: An enzyme that catalyzes the transfer of a phosphate group from one molecule to another. Kinases play a role in the regulation of cell proliferation, differentiation, metabolism, migration, and survival. A “tyrosine kinase” transfers phosphate groups to a hydroxyl group of a tyrosine in a polypeptide. In some examples, a kinase is a JAK2 kinase.

Receptor protein tyrosine kinases (PTKs) contain a single polypeptide chain with a transmembrane segment. The extracellular end of this segment contains a high affinity ligand-binding domain, while the cytoplasmic end comprises the catalytic core and the regulatory sequences.

Non-receptor tyrosine kinases, such as JAK2, can be located in the cytoplasm as well as in the nucleus. They exhibit distinct kinase regulation, substrate phosphorylation, and function.

A “preferential” inhibition of a kinase refers to decreasing activity of one kinase, such as JAK2, more than inhibiting the activity of a second kinase, such as a mitogen-activated protein kinase (MAPK) or another JAK kinase.

Label: An agent capable of detection, for example by spectrophotometry, flow cytometry, or microscopy. For example, a label can be attached to a nucleotide, thereby permitting detection of the nucleotide, such as detection of the nucleic acid molecule of which the nucleotide is a part, such as a JAK2 specific probe or primer. Labels can also be attached to antibodies, such as a TNF-alpha antibody. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Myeloproliferative disease or Myeloproliferative disorder: Non-lymphoid dysplastic or neoplastic conditions arising from a haematopoietic stem cell or its progeny. “MPD patient” includes a patient who has been diagnosed with an MPD. “Myeloproliferative disease” encompasses the specific, classified types of myeloproliferative diseases including polycythemia vera (PV), essential thrombocythemia (ET) and idiopathic myelofibrosis (IMF). Also included are hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, and systemic mastocytosis (SM) chronic myelogenous leukemia (CML), and chronic eosinophilic leukemia (CEL). “Myeloproliferative disease” also encompasses any unclassified myeloproliferative diseases (UMPD or MPD-NC).

Nucleotide: “Nucleotide” includes, but is not limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. A nucleotide sequence refers to the sequence of bases in a polynucleotide.

Oligonucleotide: An oligonucleotide is a plurality of joined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 bases long, or from about 6 to about 50 bases, for example about 10-25 bases, such as 12, 15 or 20 bases. In some examples an oligonucleotide is a probe or primer for use in detecting a JAK2 nucleic acid or a mutation thereof, such as a mutation that results in the substitution of a phenylalanine for valine at position 617 of the human JAK2 amino acid sequence.

Pharmaceutically acceptable carriers: The pharmaceutically acceptable carriers of use are conventional. Remington's Pharmaceutical Sciences, by E. W. Martin, Mack Publishing Co., Easton, Pa., 15th Edition, 1975, describes compositions and formulations suitable for pharmaceutical delivery of the compositions disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polymerizing agent: A compound capable of reacting monomer molecules (such as nucleotides) together in a chemical reaction to form linear chains or a three-dimensional network of polymer chains. A particular example of a polymerizing agent is polymerase, an enzyme, which catalyzes the 5′ to 3′ elongation of a primer strand complementary to a nucleic acid template. Examples of polymerases that can be used to amplify a nucleic acid molecule include, but are not limited to the E. coli DNA polymerase I, specifically the Klenow fragment which has 3′ to 5′ exonuclease activity, Taq polymerase, reverse transcriptase (such as HIV-1 RT), E. coli RNA polymerase, and wheat germ RNA polymerase II.

The choice of polymerase is dependent on the nucleic acid to be amplified. If the template is a single-stranded DNA molecule, a DNA-directed DNA or RNA polymerase can be used; if the template is a single-stranded RNA molecule, then a reverse transcriptase (such as an RNA-directed DNA polymerase) can be used. In some examples, a polymerizing agent is used to amplify a JAK2 nucleic acid or a TNF-alpha nucleic acid, such as is a sample obtained from a subject, to detect a mutation in the JAK2 kinase.

Polypeptide: Any chain of amino acids, regardless of length or post-translational modification (such as glycosylation, methylation, ubiquitination, phosphorylation, or the like). In one embodiment, a polypeptide is a JAK2 polypeptide. In another embodiment, a polypeptide is a TNF-alpha polypeptide. A polypeptide has an amino terminal (N-terminal) end and a carboxy terminal (C-terminal) end. “Polypeptide” is used interchangeably with peptide or protein, and is used to refer to a polymer of amino acid residues. A “residue” refers to an amino acid or amino acid mimetic incorporated in a polypeptide by an amide bond or amide bond mimetic.

Probes and primers: A probe comprises an isolated nucleic acid capable of hybridizing to a target nucleic acid (such as a JAK2 nucleic acid molecule or a TNF-alpha nucleic acid). A detectable label or reporter molecule can be attached to a probe. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example in Sambrook et al. (In Molecular Cloning: A Laboratory Manual, CSHL, New York, 1989) and Ausubel et al. (In Current Protocols in Molecular Biology, John Wiley & Sons, New York, 1998).

Probes are generally at least 12 nucleotides in length, such as at least 12, at least 13, at least 14, at least 15, at least 16, at least 17, at least 18, at least 19, least 20, at least 21, at least 22, at least 23, at least 24, at least 25, at least 26, at least 27, at least 28, at least 29, at least 30, or more contiguous nucleotides complementary to the target nucleic acid molecule, such as 12-30 nucleotides, 15-30 nucleotides, 20-30 nucleotides, or 12-29 nucleotides.

Primers are short nucleic acid molecules, for instance DNA oligonucleotides 10 nucleotides or more in length, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule (such as a portion of a JAK2 nucleic acid molecule or a TNF-alpha nucleic acid).

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 15, 20, 25, 30, 35, 40, 45, 50 or more consecutive nucleotides. In particular examples, a primer is at least 15 nucleotides in length, such as at least 15 contiguous nucleotides complementary to a target nucleic acid molecule. Particular lengths of primers that can be used to practice the methods of the present disclosure (for example, to amplify a region of a JAK2 nucleic acid molecule) include primers having at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 15-50 nucleotides, 20-50 nucleotides, or 15-30 nucleotides.

Primer pairs can be used for amplification of a nucleic acid sequence, for example, by PCR, real-time PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction.

PCR primer pairs can be derived from a known sequence (such as the JAK2 nucleic acid molecule encoding the JAK2 amino acid sequence as set forth in SEQ ID NO: 1, or 3) for example, by using computer programs intended for that purpose such as Primer (Version 0.5, © 1991, Whitehead Institute for Biomedical Research, Cambridge, Mass.) or PRIMER EXPRESS® Software (Applied Biosystems, AB, Foster City, Calif.).

Sample: A sample, such as a biological sample, is a sample obtained from a plant or animal subject. As used herein, biological samples include all clinical samples useful for detection of TNF-alpha and/or JAK2 in subjects, including, but not limited to, cells, tissues, and bodily fluids, such as: blood; derivatives and fractions of blood, such as serum; extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; pus; or bone marrow aspirates. In particular embodiments, the biological sample is obtained from a subject, such as in the form of blood or serum.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Homologs and variants of a JAK2 protein are typically characterized by possession of at least 50% sequence identity counted over the full length alignment with the amino acid sequence of a native protein using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 60%, at least 65%, at least 70%, at least 75%, at least 80%, at least 90%, at least 95%, at least 96%, at least 97%, at least 98%, or at least 99% sequence identity. When less than the entire sequence is being compared for sequence identity, homologs and variants will typically possess at least 75% sequence identity over short windows of 10-20 amino acids, and may possess sequence identities of at least 85% or at least 90% at least 95%, at least 96%, at least 97%, at least 98%, or at least 99% depending on their similarity to the reference sequence. Methods for determining sequence identity over such short windows are described at the NCBI website. One of skill in the art will appreciate that these sequence identity ranges are provided for guidance only; it is entirely possible that strongly significant homologs could be obtained that fall outside of the ranges provided.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166=1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. One indication that two nucleic acid molecules are closely related is that the two molecules hybridize to each other under stringent conditions.

Tissue Necrosis Factor alpha (TNF-alpha or TNF alpha): TNF-alpha is primarily secreted by macrophages and exerts pro-inflammatory activity. Local activity of TNF-alpha helps to contain infection, however, when infection spreads to the blood and there is systemic release of TNF-alpha, septic shock and/organ failure can occur due to loss of plasma volume as a result of increased vascular permeability. TNF-alpha found in vivo in humans as a 17 kDa homotrimeric protein with subunits having 157 amino acids, and variants thereof. Human and mouse TNF-alpha have approximately 80% identity at the amino acid level. The human TNF-alpha gene maps to chromosome 6p21.3, has 4 exons, and spans approximately 3 kb. It is understood that TNF-alpha includes both naturally occurring and recombinant TNF-alpha peptides, as well as TNF-alpha fragments and TNF-alpha variants that retain full or partial IL-15 biological activity.

TNF-alpha sequences are publicly available. For example, GENBANK® Accession number NM_{—}013693 discloses a mouse TNF-alpha gene sequence, and GENBANK® Accession numbers NP_{—}038721 disclose a mouse TNF-alpha protein sequence. In an other examples, GENBANK® Accession number NM_{—}000594 discloses a human TNF-alpha gene sequence, and GENBANK® Accession number NP_{—}000585 disclose a human TNF-alpha protein sequence. One skilled in the art will appreciate that TNF-alpha nucleic acid and protein molecules can vary from those publicly available, such as those having one or more substitutions, deletions, insertions, or combinations thereof, while still retaining TNF-alpha biological activity.

TNF-alpha inhibitors include small molecule inhibitors, inhibitory nucleic acids such as siRNA, antisense molecules and ribozymes, and antibodies that specifically bind TNF-alpha. Exemplary TNF-alpha inhibitors that are antibodies include Adalimumab (HUMIRA™), Etanercept (ENBREL®), Certolizumab pegol (CIMZIA®), Golimumab, Nerelimomaband Infliximab (REMICADE®). Exemplary small molecule inhibitors targeting TNF-alpha are LMP-160, LMP-420, (LEUKOMED INC.).

Therapeutically effective amount: The quantity of a composition, such as a TNF-alpha inhibitor, sufficient to achieve a desired effect in a subject being treated. For instance, this can be the amount necessary to inhibit the progression of a myeloproliferative disorder or to measurably alter outward symptoms of the myeloproliferative disorder. When administered to a subject, a dosage will generally be used that will achieve target tissue concentrations that has been shown to achieve in vitro inhibition of or to measurably alter outward symptoms of a myeloproliferative disorder.

Wild-type: A gene or a gene product that has the characteristics of that gene or gene product when isolated from a naturally occurring source. A wild-type gene is that which is most frequently observed in a population and is thus arbitrarily designated the “normal” or “wild-type” form of the gene. “Wild-type” can also refer to the sequence at a specific nucleotide position or positions, or the sequence at a particular codon position or positions, or the sequence at a particular amino acid position or positions. For example, a JAK2 gene can be “wild-type” at nucleotide position 1849 or at codon 617. As used herein, “mutant,” “modified” or “polymorphic” refers to a gene or gene product which displays modifications in sequence and/or functional properties (altered characteristics, such as kinase activity) when compared to the wild-type gene or gene product. “Mutant,” “modified” or “polymorphic” also refers to the sequence at a specific nucleotide position or positions, or the sequence at a particular codon position or positions, or the sequence at a particular amino acid position or positions.

### II. Description of Several Embodiments

Certain neoplastic diseases including non-CML myeloproliferative diseases (MPDs) such as polycythemia vera (PV), essential thrombocythemia (ET), and chronic idiopathic myelofibrosis (IMF) and as of yet unclassified myeloproliferative diseases (MPD-NC) are characterized by an aberrant increase in blood cells (See Vainchenker and Constantinescu, Hematology (American Society of Hematology), 195-200, 2005). This increase is generally initiated by a spontaneous mutation in a multipotent hematopoetic stem cell located in the bone marrow. Due to the mutation, the stem cell produces far more blood cells of a particular lineage than normal, resulting in the overproduction of cells such as erythroid cells, megakaryocytes, granulocytes and monocytes. Some symptoms common to patients with MPD include enlarged spleen, enlarged liver, elevated white, red and/or platelet cell count, blood clots (thrombosis), weakness, dizziness and headache. Diseases such as PV, ET and IMF may presage leukemia, however the rate of transformation (e.g., to blast crisis) differs with each disease.

A mutation in the Janus Activating kinase 2 (JAK2) gene (see Genbank®Accession No. NM004972, herein incorporated by reference in its entirety), a cytoplasmic, nonreceptor tyrosine kinase, has been identified in a number of MPDs. For example, this mutation has been reported in up to 97% of patients with PV, and in greater than 40% of patients with either ET or IMF.

In this JAK2 mutant, a valine (codon “GTC”) is replaced by a phenylalanine (codon “TTC”) at amino acid position 617 (the “V617F mutant”) (Baxter et al., Lancet 365:1054-1060, 2005). Amino acid 617 is located in exon 12 which includes a pseudokinase, auto-inhibitory (or negative regulatory) domain termed JH2 (Jak Homology 2 domain). Though this domain has no kinase activity, it interacts with the JH1 (Jak Homology 1) domain, which does have kinase activity (Baxter et al., Lancet 365:1054-1060, 2005). Appropriate contact between the two domains in the wild-type protein allows proper kinase activity and regulation. However, the V617F mutation causes improper contact between the two domains, resulting in constitutive kinase activity in the mutant JAK2 protein. A variety of different approaches and a large body of evidence suggest that, when present, the JAK2 V617F mutation contributes to the pathogenesis of MPD and homozygous and heterozygous cell populations have been reported in MPD patients.

However, it was not known prior to this disclosure that TNF-alpha signaling was a component of JAK2 induced myeloproliferative disorders. Disclosed herein is the surprising discovery that, cellular proliferative disorders caused by JAK2 activating mutations depend on the presence of the hormone Tumor Necrosis Factor (TNF)-alpha to grow and cause the MPD. This discovery was based on the observation that mice with normal TNF-alpha production and an activating JAK2 mutation developed MPS very quickly, while mice that lack TNF-alpha do not develop MPD. Thus, inhibition of TNF-alpha represents a novel approach to the treatment of myeloproliferative disorders.

### A. Methods of Treatment

Methods for treating a subject with a cellular proliferative disorder are provided herein. The methods include selecting an individual that has an activating mutation in a JAK2 kinase (for example using the methods described in Section C). Typical subjects intended for treatment with an inhibitor of JAK2 kinase activity and an inhibitor of TNF-alpha activity include humans, as well as non-human primates and other animals, such as mice.

After selection, the subject is administered a therapeutically effective amount of an inhibitor of JAK2 kinase activity and a therapeutically effective amount of an inhibitor of TNF-alpha activity, thereby treating the cellular proliferative disorder. In some examples, the inhibitor of JAK2 kinase activity and the inhibitor of TNF-alpha activity are provided as a pharmaceutical composition or compositions (see Section B).

In several embodiments, the cellular proliferative disorder is a myeloproliferative disorder, such as one or more of polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, systemic mastocytosis (SM), unclassified myeloproliferative disease (UMPD or MPD-NC), chronic myelogenous leukemia (CML), and chronic eosinophilic leukemia (CEL). In some embodiments, a cellular proliferative disorder is AML such as AML-FAB M7.

Agents that inhibit TNF-alpha activity include agents that inhibit the expression and/or signaling activity of TNF-alpha, for example, small molecule inhibitors, inhibitory nucleic acids such as siRNA, antisense molecules and ribozymes, and antibodies that specifically bind TNF-alpha. Exemplary TNF-alpha inhibitors that are antibodies include Adalimumab (HUMIRA™), Etanercept (ENBREL®), Certolizumab pegol (CIMZIA®), Golimumab, Nerelimomab and Infliximab (REMICADE®). In some embodiments, a subject is administered one or more antibodies that specifically bind TNF-alpha, such as one or more of Adalimumab, Etanercept, Certolizumab pegol, Golimumab, Nerelimomab and Infliximab. Exemplary small molecule inhibitors targeting TNF-alpha are LMP-160 and LMP-420, (LEUKOMED INC.). In some embodiments, a subject is administered one or more small molecule inhibitors of TNF-alpha, such as one or more of LMP-160 and LMP-420. In some embodiments, a subject is administered one or more small molecule inhibitors of TNF-alpha and one or more or antibodies that specifically bind TNF-alpha.

Exemplary JAK2 inhibitors are antibodies that specifically bind JAK2, siRNAs, ribozymes, antisense molecules, and small molecule kinase inhibitors, such as Lestaurtinb (CEP701, CEPHALON®), TG101348 (TargeGen, Inc.), CYT387 (Cytopia), AZ960 (AstraZeneca), SGI-1252 (SUPERGE®), WP1066, AG490 (A.G. Scientific Inc.), INCB18424 (Incyte), and SB1518 (S*BIO™). In some embodiments, a subject is administered one or more small molecule kinase inhibitors of JAK2 kinase activity, such as one or more of Lestaurtinb, TG101348, CYT387, AZ960, SGI-1252, WP1066, AG490, INCB18424, and SB1518.

The administration of the inhibitors of TNF-alpha activity and inhibitors of JAK2 kinase activity can be for either prophylactic or therapeutic purpose. When provided prophylactically, the inhibitors of TNF-alpha activity and inhibitors of JAK2 kinase activity are provided in advance of any symptom. The prophylactic administration of the compounds serves to prevent or ameliorate any subsequent disease process. When provided therapeutically, the compounds are provided at (or shortly after) the onset of a symptom of disease.

For prophylactic and therapeutic purposes, the inhibitors of TNF-alpha activity and inhibitors of JAK2 kinase activity can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition.

Determination of effective dosages is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the inhibitors of TNF-alpha activity and inhibitors of JAK2 kinase activity (for example, amounts that are effective to alleviate one or more symptoms of a targeted disease or condition). In alternative embodiments, an effective amount or effective dose of the inhibitors of TNF-alpha activity and inhibitors of JAK2 kinase activity may simply inhibit or enhance one or more selected biological activities correlated with a disease or condition.

The actual dosage of inhibitors of TNF-alpha activity and inhibitors of JAK2 kinase activity will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the inhibitors of TNF-alpha activity and inhibitors of JAK2 kinase activity for eliciting the desired activity or biological response in the subject. Dosage regimens can be adjusted to provide an optimum prophylactic or therapeutic response.

A therapeutically effective amount is also one in which any toxic or detrimental side effects of the compound and/or other biologically active agent is outweighed in clinical terms by therapeutically beneficial effects. A non-limiting range for a therapeutically effective amount of a inhibitors of TNF-alpha activity and inhibitors of JAK2 kinase activity within the methods and formulations of the disclosure is about 0.0001 μg/kg body weight to about 10 mg/kg body weight per dose, such as about 0.0001 μg/kg body weight to about 0.001 μg/kg body weight per dose, about 0.001 μg/kg body weight to about 0.01 μg/kg body weight per dose, about 0.01 μg/kg body weight to about 0.1 μg/kg body weight per dose, about 0.1 μg/kg body weight to about 10 μg/kg body weight per dose, about 1 μg/kg body weight to about 100 μg/kg body weight per dose, about 100 μg/kg body weight to about 500 μg/kg body weight per dose, about 500 μg/kg body weight per dose to about 1000 μg/kg body weight per dose, or about 1.0 mg/kg body weight to about 10 mg/kg body weight per dose.

Dosage can be varied by the attending clinician to maintain a desired concentration at a target site. Higher or lower concentrations can be selected based on the mode of delivery, for example, trans-epidermal, rectal, oral, pulmonary, intranasal delivery, intravenous or subcutaneous delivery. To achieve the same serum concentration level, for example, slow-release particles with a release rate of 5 nanomolar (under standard conditions) would be administered at about twice the dosage of particles with a release rate of 10 nanomolar.

The specific dose level and frequency of dosage for any particular subject may be varied and will depend upon a variety of factors, including the activity of the specific compound, the extent of existing disease activity, the age, body weight, general health, sex, diet, mode and time of administration, rate of excretion, drug combination, and severity of the condition of the host undergoing therapy.

### B. Pharmaceutical Compositions

Compositions, such as therapeutic or pharmaceutical compositions, are provided that include an inhibitor of TNF-alpha activity and/or an inhibitor of JAK2 kinase activity. It is desirable to prepare the inhibitors of TNF-alpha activity and/or the inhibitors of JAK2 kinase activity as a pharmaceutical composition appropriate for the intended application, for example to inhibit or treat a cellular proliferative disorder. Accordingly, methods for making a medicament or pharmaceutical composition containing an inhibitor of TNF-alpha activity and/or an inhibitor of JAK2 kinase activity are included herein Inhibitors of TNF-alpha activity and/or inhibitors of JAK2 kinase activity can be prepared for administration alone or with other active ingredients, such as other chemotherapeutics.

When an inhibitor of TNF-alpha activity and an inhibitor of JAK2 kinase activity are administered to a subject, the administration can be concurrent or sequential. Sequential administration can be separated by any amount of time, so long as the desired affect is achieved. Multiple administrations of the compositions described herein are also contemplated.

Pharmaceutical compositions including an inhibitor of TNF-alpha activity and/or an inhibitor of JAK2 kinase activity can be administered to subjects by a variety of routes. This includes oral, nasal (such as intranasal), ocular, buccal, enteral, intravitral, or other mucosal (such as rectal or vaginal) or topical administration. Alternatively, administration will be by orthotopic, intradermal subcutaneous, intramuscular, parentral intraperitoneal, or intravenous injection routes. Such pharmaceutical compositions are usually administered as pharmaceutically acceptable compositions that include physiologically acceptable carriers, buffers or other excipients.

Typically, preparation of a pharmaceutical composition (for example, for use as a medicament or in the manufacture of a medicament) entails preparing a pharmaceutical composition that is essentially free of pyrogens, as well as any other impurities that could be harmful to humans or animals. The inhibitors of TNF-alpha activity and/or an inhibitors of JAK2 kinase activity may be included in pharmaceutical compositions (including therapeutic and prophylactic formulations), which are typically combined together with one or more pharmaceutically acceptable vehicles or carriers and, optionally, other therapeutic ingredients.

To formulate the pharmaceutical compositions, the inhibitor of TNF-alpha and/or the inhibitor of JAK2 kinase activity can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80), solubility enhancing agents (for example, cyclodextrins and derivatives thereof), stabilizers (for example, serum albumin), and reducing agents (for example, glutathione) can be included. Adjuvants, such as aluminum hydroxide (for example, Amphogel, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The inhibitor of TNF-alpha activity and/or the inhibitor of JAK2 kinase activity can be can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly (hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, and microspheres.

The inhibitor of TNF-alpha activity and/or the inhibitor of JAK2 kinase activity can be can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The inhibitor of TNF-alpha activity and/or the inhibitor of JAK2 kinase activity can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the inhibitor of TNF-alpha activity and/or the inhibitor JAK2 kinase activity can be also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

Antibodies may be provided in lyophilized form and rehydrated with sterile water before administration, although they are also provided in sterile solutions of known concentration. In some examples, the antibody solution is then added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of RITUXAN® in 1997. Antibodies can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

For prophylactic and therapeutic purposes, the pharmaceutical compositions can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein.

Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of an inhibitor of TNF-alpha activity or an inhibitor of JAK2 kinase activity.

The appropriate dose will vary depending on the characteristics of the subject, for example, whether the subject is a human or non-human, the age, weight, and other health considerations pertaining to the condition or status of the subject, the mode, route of administration, and number of doses, and whether the pharmaceutical composition includes both inhibitor of TNF-alpha activity or an inhibitor of JAK2 kinase activity, time and route of administration, other drugs or treatments being administered concurrently, as well as the specific pharmacology of the therapeutic compositions for eliciting the desired activity or biological response in the subject. Dosage regimens can be adjusted to provide an optimum prophylactic or therapeutic response.

A therapeutically effective amount is also one in which any toxic or detrimental side effects of the compound and/or other biologically active agent is outweighed in clinical terms by therapeutically beneficial effects. A non-limiting range for a therapeutically effective amount of an inhibitor of TNF-alpha activity or an inhibitor of JAK2 kinase activity within the methods and formulations of the disclosure is about 0.0001 μg/kg body weight to about 10 mg/kg body weight per dose, such as about 0.0001 μg/kg body weight to about 0.001 μg/kg body weight per dose, about 0.001 μg/kg body weight to about 0.01 μg/kg body weight per dose, about 0.01 μg/kg body weight to about 0.1 μg/kg body weight per dose, about 0.1 μg/kg body weight to about 10 μg/kg body weight per dose, about 1 μg/kg body weight to about 100 μg/kg body weight per dose, about 100 μg/kg body weight to about 500 μg/kg body weight per dose, about 500 μg/kg body weight per dose to about 1000 μg/kg body weight per dose, or about 1.0 mg/kg body weight per dose to about 10 mg/kg body weight per dose.

Therapeutic compositions that include an inhibitor of TNF-alpha activity or an inhibitor of JAK2 kinase activity can be delivered by way of a pump (see Langer, supra; Sefton, CRC Crit. Ref Biomed. Eng. 14:201, 1987; Buchwald et al., Surgery 88:507, 1980; Saudek et al., N. Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer (Science 249:1527-33, 1990).

In one example, a pump is implanted (for example see U.S. Pat. Nos. 6,436,091; 5,939,380; and 5,993,414). Implantable drug infusion devices are used to provide patients with a constant and long-term dosage or infusion of a therapeutic agent. Such device can be categorized as either active or passive.

Active drug or programmable infusion devices feature a pump or a metering system to deliver the agent into the patient's system. An example of such an active infusion device currently available is the Medtronic SYNCHROMED™programmable pump. Passive infusion devices, in contrast, do not feature a pump, but rather rely upon a pressurized drug reservoir to deliver the agent of interest. An example of such a device includes the Medtronic ISOMED™.

In particular examples, therapeutic compositions are administered by sustained-release systems. Suitable examples of sustained-release systems include suitable polymeric materials (such as, semi-permeable polymer matrices in the form of shaped articles, for example films, or mirocapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides (U.S. Pat. No. 3,773,919, EP 58,481), copolymers of L-glutamic acid and gamma-ethyl-L-glutamate (Sidman et al., Biopolymers 22:547-556, 1983, poly(2-hydroxyethyl methacrylate)); (Langer et al., J. Biomed. Mater. Res. 15:167-277, 1981; Langer, Chem. Tech. 12:98-105, 1982, ethylene vinyl acetate (Langer et al., Id.) or poly-D-(−)-3-hydroxybutyric acid (EP 133,988).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (for example, U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

### C. Method for Determining if a Subject is Susceptible to Treatment

Methods are provided herein for determining if a subject with a cellular proliferative disorder would benefit from treatment with an agent that inhibits tumor TNF-alpha, for example to select a subject for treatment with an inhibitor of TNF-alpha activity and an inhibitor of JAK2 kinase activity. The methods include detecting the presence of an activating mutation in JAK2 in a biological sample from the subject, for example by detecting a mutation in the JAK2 protein or a nucleic acid encoding the protein. The presence of the activating mutation in JAK2 indicates that the subject will benefit from treatment with the agent that inhibits TNF-alpha.

In several embodiments, the cellular proliferative disorder is a myeloproliferative disorder, such polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia (CEL), systemic mastocytosis (SM), unclassified myeloproliferative disease (UMPD or MPD-NC), or chronic myelogenous leukemia (CML). In some embodiments, a cellular proliferative disorder is AML such as AML-FAB M7. Such a subject is selected for treatment with an inhibitor of TNF-alpha activity and JAK2 kinase activity used the methods of treatment disclosed in Section A above.

Methods for isolating nucleic acids, and determining the presence of an activating mutation in JAK2 are known in the art, and are described for example, in U.S. Patent Application Publication No. 2006/0288432 and U.S. Patent Application Publication No. 2007/0248961, which are herein incorporated by reference. Both of these documents describe specific assays of use to detect the V617F mutant, wherein a valine is replace by a phenylalanine at position 617.

Any biological sample is of use, including but not limited to, blood, serum, plasma, bone marrow biopsy or sputum samples. Methods of plasma and serum preparation are well known in the art. Either “fresh” blood plasma or serum, or frozen (stored) and subsequently thawed plasma or serum may be used. Frozen (stored) plasma or serum can be maintained at storage conditions of −20 to −70 degrees centigrade until thawed and used. “Fresh” plasma or serum can be refrigerated or maintained on ice until used, with nucleic acid (RNA, DNA or total nucleic acid) extraction being performed as soon as possible.

Blood can be drawn by standard methods into a collection tube, preferably siliconized glass, either without anticoagulant for preparation of serum, or with EDTA, sodium citrate, heparin, or similar anticoagulants for preparation of plasma. The preferred method if preparing plasma or serum for storage, although not an absolute requirement, is that plasma or serum be first fractionated from whole blood prior to being frozen. This reduces the burden of extraneous intracellular RNA released from lysis of frozen and thawed cells which might reduce the sensitivity of the amplification assay or interfere with the amplification assay through release of inhibitors to PCR such as porphyrins and hematin. “Fresh” plasma or serum may be fractionated from whole blood by centrifugation, using preferably gentle centrifugation at 300-800 times gravity for five to ten minutes, or fractionated by other standard methods. Since heparin may interfere with RT-PCR, use of heparinized blood may require pretreatment with heparinase, followed by removal of calcium prior to reverse transcription (Imai, H., et al., J. Virol. Methods 36:181-184, 1992). Numerous methods are known in the art for isolating total nucleic acid, DNA and RNA from blood, serum, plasma and bone marrow or other hematopoietic tissues.

In some examples, total nucleic acid can be extracted from patient plasma or peripheral blood cells. In other methods, mRNA can be extracted from patient blood/bone marrow samples. General methods for mRNA extraction are well known in the art and are disclosed in standard textbooks of molecular biology, including Ausubel et al., Current Protocols of Molecular Biology, John Wiley and Sons (1997). Previously described methods, kits or systems for extraction of mammalian RNA or viral RNA may be adapted, either as published or modified for the extraction of tumor-derived or associated RNA from plasma or serum. RNA can be extracted from plasma or serum using silica particles, glass beads, or diatoms (see Boom, R., et al., J. Clin. Micro. 28:495-503, 1990). In particular, RNA isolation can be performed using purification kit, buffer set and protease from commercial manufacturers, such as QIAGEN®, according to the manufacturer's instructions. For example, total RNA from cells in culture (such as those obtained from a subject) can be isolated using QIAGEN® RNeasy® mini-columns. Other commercially available RNA isolation kits include MASTERPURE®. Complete DNA and RNA Purification Kit (EPICENTRE® Madison, Wis.), and Paraffin Block RNA Isolation Kit (Ambion®, Inc.). Total RNA from tissue samples can be isolated using RNA Stat-60 (Tel-Test). RNA prepared from tumor or other biological sample can be isolated, for example, by cesium chloride density gradient centrifugation. As an alternative method, RNA can be extracted from using the Acid Guanidinium Thiocyanate-Phenol-chloroform extraction method (Chomczynski, P. and Sacchi, N., Analytical Biochemistry 162:156-159, 1987).

Nucleic acid extracted from tissues, cells, plasma or serum can be amplified using nucleic acid amplification techniques well know in the art. Many of these amplification methods can also be used to detect the presence of mutations simply by designing oligonucleotide primers or probes to interact with or hybridize to a particular target sequence in a specific manner. By way of example, but not by way of limitation these techniques can include the polymerase chain reaction (PCR) reverse transcriptase polymerase chain reaction (RT-PCR), nested PCR, ligase chain reaction. See Abravaya, K., et al., Nucleic Acids Research 23:675-682, (1995), branched DNA signal amplification, Urdea, M. S., et al., AIDS 7 (suppl 2):S11-S14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods 35:273-286, (1991), Invader Technology, or other sequence replication assays or signal amplification assays.

In one non-limiting example, a PCR reaction can be performed using either the total nucleic acid preparation or the RNA preparation to specifically amplify a portion of the patient RNA. An exemplary one-step RT-PCR system is the SUPERSCRIPT III® System (Invitrogen, Carlsbad, Calif.). Other methods and systems for RT-PCR reactions are well known in the art and are commercially available. A primer pair is designed to encompass a region of interest, for example, the V617F mutation in JAK2 nucleic acid, to yield a PCR product. By way of example, but not by way of limitation, a primer pair for JAK2 may be 5′-GAC TAC GGT CAA CTG CAT GAA A-3′ (SEQ ID NO: 9), and 5′-CCA TGC CAA CTG TTT AGC AA-3′ (SEQ ID NO: 10). The resulting RT-PCR product is 273 nucleotides long. The RT-PCR product can then be purified, for example by gel purification, and the resulting purified product can be sequenced. Nucleic acid sequencing methods are known in the art; an exemplary sequencing method includes the ABI Prism BIGDYE® Terminator v3.1 Cycle Sequencing Kit (Applied Biosystems, Foster City, Calif.). The sequencing data can then be analyzed for the presence or absence of one or more activating mutations in JAK2 nucleic acid. The sequencing data can also be analyzed to determine the proportion of wild-type to mutant nucleic acid present in the sample.

The presence of one or more activating mutations in JAK2, such as a V617F mutation, indicates that an agent that inhibits TNF-alpha expression and/or activity is of use for the treatment of the subject. Agents that inhibit TNF-alpha expression and/or activity include, for example, small molecule inhibitors, inhibitory nucleic acids such as siRNA, antisense molecules and ribozymes, and antibodies that specifically bind TNF-alpha. Exemplary TNF-alpha inhibitors that are antibodies include Adalimumab (HUMIRA™), Etanercept (ENBREL®), Certolizumab pegol (CIMZIA®), Golimumab, Nerelimomaband Infliximab (REMICADE®). Exemplary small molecule inhibitors targeting TNF-alpha are LMP-160, LMP-420, (LEUKOMED INC.). Exemplary JAK2 inhibitors are antibodies that specifically bind JAK2, siRNAs, ribozymes, antisense molecules, and kinase inhibitors, such as Lestaurtinb (CEP701, CEPHALON®), TG1013418 (TargeGen, Inc.), CYT387 (Cytopia), AZ960 (AstraZeneca), SGI-1252 (SUPERGE®), WP1066, AG490 (A.G. Scientific Inc.), INCB18424 (Incyte), and SB1518 (S*BIO™)

### D. Screening Methods

Methods are provided herein for identifying an agent of use in treating a subject with a cellular proliferative disorder, such as a myeloproliferative disorder and/or cancer, or with a predisposition for cellular proliferative disorder. The methods include contacting an isolated cell expressing an activating mutation in the JAK2 protein with a test agent, and detecting the amount of tumor necrosis TNF-alpha produced by the cell. The amount of TNF-alpha produced by the cell is compared to a control. A reduction in the amount of TNF-alpha produced by the cell relative to the control indicates that the agent as useful for the treatment of a subject with the cellular proliferative disorder or with a predisposition for developing the cellular proliferative disorder. The isolated cell can be any cell of interest, including human and non-human cells. For example, the cells can be mouse, rat, monkey, or human cells. In some embodiments, the activating mutation in the JAK2 protein is a valine to phenylalanine mutation at position 617 of SEQ ID NO: 1 or SEQ ID NO: 3.

In several embodiments, the cellular proliferative disorder is one or more of polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia (CEL), systemic mastocytosis (SM), unclassified myeloproliferative disease (UMPD or MPD-NC), or chronic myelogenous leukemia (CML). In some embodiments, the cellular proliferative disorder is AML such as AML-FAB M7.

A variety of controls can be used in these methods. In some embodiments, the control is a standard value. In other embodiments, the control is the amount of TNF-alpha produced by an isolated cell expressing the activating mutation in the JAK2 protein not contacted with the agent or contacted with an agent known not to affect Jak2. Detecting expression of TNF-alpha can include detecting the amount of an mRNA encoding TNF-alpha, detecting the amount of TNF-alpha protein, or both.

In one embodiment, for a high throughput format, cells can be introduced into wells of a multiwell plate or of a glass slide or microchip, and can be contacted with the test agent. Generally, the cells are organized in an array, particularly an addressable array, such that robotics conveniently can be used for manipulating the cells and solutions and for monitoring the cells, particularly with respect to the function being examined, such as TNF-alpha expression. An advantage of using a high throughput format is that a number of test agents can be examined in parallel, and, if desired, control reactions also can be run under identical conditions as the test conditions. As such, the methods disclosed herein provide a means to screen one, a few, or a large number of test agents in order to identify an agent that can alter a function of cells, for example, an agent that inhibits TNF-alpha expression.

In additional embodiments, methods are provided for identifying an agent of use for treating a cellular proliferative disorder, such as cancer and/or a myeloproliferative disorder, utilizing an animal model system. These methods include transplanting a bone marrow host cell comprising a nucleic acid encoding a JAK2 mutant into a non-human mammal, wherein the JAK2 mutant is has a valine codon replaced by a phenylalanine codon at amino acid position 617, and wherein the bone marrow host cell comprises a wild-type gene encoding TNF-alpha. In some examples, the non-human mammal is immunocompromised, such as a non-human mammal treated with gamma-irradiation or with a genetic immunodeficiency. For example, the non-human mammal can be a sub-lethally irradiated mouse or a severe combined immunodeficiency (SCID) mouse.

The non-human mammal is treated with an agent of interest, and the expression of TNF-alpha is detected. A decrease in the expression of TNF-alpha produced by the non-human mammal as compared to a control indicates that the agent is of use in treating the myeloproliferative disorder. The control can be a standard value. The control can also be a non-human animal transplanted with a bone marrow host cell comprising a nucleic acid encoding a JAK2 mutant, wherein the JAK2 mutant is has a valine codon replaced by a phenylalanine codon at amino acid position 617, and wherein the bone marrow host cell comprises a wild-type gene encoding TNF-alpha, wherein the non-human mammal is not treated with the agent of interest. For example, the control non-human mammal can be treated with vehicle alone or treated with an agent known not to be of use for treating the myeloproliferative disorder.

Expression of cytokines can be evaluated by detecting the presence of mRNA encoding the cytokine of interest, either qualitatively or quantitatively. Thus, in several embodiments, the disclosed methods include evaluating mRNA encoding TNF-alpha. In several examples, the mRNA can be quantitated. RNA can be isolated using methods well known to one of skill in the art.

Methods of detecting RNA encoding TNF-alpha based on hybridization analysis and/or sequencing are known in the art. The most commonly used methods known in the art for the quantification of mRNA expression in a sample include northern blotting and in situ hybridization (Parker & Barnes, Methods in Molecular Biology 106:247 283, 1999); RNAse protection assays (Hod, Biotechniques 13:852 854 (1992)); and PCR-based methods, such as reverse transcription polymerase chain reaction (RT-PCR) (Weis et al., Trends in Genetics 8:263 264, 1992). Representative methods for sequencing-based gene expression analysis include Serial Analysis of Gene Expression (SAGE), and gene expression analysis by massively parallel signature sequencing (MPSS). In one example, RT-PCR, can be used to compare mRNA levels in different samples, with or without drug treatment, to characterize patterns of gene expression, to discriminate between closely related mRNAs, and to analyze RNA structure.

Methods for quantitating mRNA are well known in the art. In one example, the method utilizes reverse transcriptase polymerase chain reaction (RT-PCR). Generally, the first step in gene expression profiling by RT-PCR is the reverse transcription of the RNA template into cDNA, followed by its exponential amplification in a PCR reaction. The two most commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP® RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® RT-PCR can be performed using commercially available equipment, such as, for example, ABI PRISM 7700® Sequence Detection System™ (Perkin-Elmer-Applied Biosystems, Foster City, Calif., USA), or Lightcycler (Roche Molecular Biochemicals, Mannheim, Germany). In one embodiment, the 5′ nuclease procedure is run on a real-time quantitative PCR device such as the ABI PRISM 7700® Sequence Detection System. The system includes of thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

In some examples, 5′-nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR can be performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH), beta-actin, and 18S ribosomal RNA.

An alternative quantitative nucleic acid amplification procedure is described in U.S. Pat. No. 5,219,727, which is incorporated herein by reference. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In additional embodiments gene expression can also be identified, or confirmed using the microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. In this manner, the expression of TNF-alpha can be evaluated along with the expression of additional molecules of interest, such as other cytokines.

In several embodiments, the amount of TNF-alpha protein is assessed. A decrease in the amount TNF-alpha proteins indicates that the agent of interest is of use to treat a myeloproliferative disorder.

The availability of antibodies specific to TNF-alpha proteins facilitates detection and/or quantitation using an number immunoassay methods that are well known in the art, such as those presented in Harlow and Lane (Antibodies, A Laboratory Manual, CSHL, New York, 1988). Methods of constructing such antibodies are known in the art. It should be noted that antibodies to all of these proteins are available from several commercial sources.

Any standard immunoassay format (such as ELISA, Western blot, or RIA assay) can be used to measure protein levels. A comparison to tissue from an organ of a cancer-free subject can easily be performed. Thus, an increase or decrease in TNF-alpha protein level can readily be evaluated using these methods. Immunohistochemical techniques can also be utilized for cytokine detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998). For the purposes of quantitating cytokine proteins, a biological sample of the subject that includes cellular proteins can be used. TNF-alpha protein can be quantiated by immunoassay.

Quantitative spectroscopic approaches methods, such as SELDI, that can be used to analyze a biological sample to determine the amount of TNF-alpha present in the sample. In one example, surface-enhanced laser desorption-ionization time-of-flight (SELDI-TOF) mass spectrometry is used to detect changes in differential protein expression, for example by using the ProteinChip™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption.

Briefly, one version of SELDI uses a chromatographic surface with a chemistry that selectively captures analytes of interest, such as cytokine proteins. Thus, this method can be used to detect TNF-alpha in addition to other cytokines Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface (such as cytokines) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array. Therefore, in a particular example, the chromatographic surface includes at least antibodies that specifically bind TNF-alpha.

In another example, antibodies, including antibodies to TNF-alpha, are immobilized onto the surface using a bacterial Fc binding support. The chromatographic surface is incubated with a sample from a subject of interest. The antigens present in the sample can recognize the antibodies on the chromatographic surface. The unbound proteins and mass spectrometric interfering compounds are washed away and the proteins that are retained on the chromatographic surface are analyzed and detected by SELDI-TOF.

Any suitable compound or composition can be used as a test agent, such as organic or inorganic chemicals, including aromatics, fatty acids, and carbohydrates; peptides, including monoclonal antibodies, polyclonal antibodies, and other specific binding agents; phosphopeptides; or nucleic acid molecules. In a particular example, the test agent includes a random peptide library (for example see Lam et al., Nature 354:82-4, 1991), random or partially degenerate, directed phosphopeptide libraries (for example see Songyang et al., Cell 72:767-78, 1993). A test agent can also include a complex mixture or “cocktail” of molecules.

Test agents can be pharmacologic agents already known in the art or can be compounds previously unknown to have any pharmacological activity. The compounds can be naturally occurring or designed in a laboratory. They can be isolated from microorganisms, animals, or plants, and can be produced recombinantly, or synthesized by chemical methods known in the art. If desired, test substances can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds (for example see Lam, Anticancer Drug Des. 12:145, 1997).

Methods for the synthesis of molecular libraries are well known in the art (see, for example, DeWitt et al., Proc. Natl. Acad. Sci. U.S.A. 90:6909, 1993; Erb et al. Proc. Natl. Acad. Sci. U.S.A. 91:11422, 1994; Zuckermann et al., J. Med. Chem. 37:2678, 1994; Cho et al., Science 261:1303, 1993; Carell et al., Angew. Chem. Int. Ed. Engl. 33:2059, 1994; Carell et al., Angew. Chem. Int. Ed. Engl. 33:2061; Gallop et al., J. Med. Chem. 37:1233, 1994). Libraries of compounds can be presented in solution (see, for example, Houghten, BioTechniques 13:412-21, 1992), or on beads (Lam, Nature 354, 82-4, 1991), chips (Fodor, Nature 364:555-6, 1993), bacteria or spores (Ladner, U.S. Pat. No. 5,223,409), plasmids (Cull et al., Proc. Natl. Acad. Sci. U.S.A. 89:1865-9, 1992), or phage (Scott & Smith, Science 249:386-90, 1990; Devlin, Science 249:404-6, 1990); Cwirla et al., Proc. Natl. Acad. Sci. 97:6378-82, 1990; Felici, J. Mol. Biol. 222:301-10, 1991; and Ladner, U.S. Pat. No. 5,223,409).

The following examples are illustrative only and are not meant to be limiting.

### EXAMPLES

### Example 1

The example describes exemplary materials and methods used in the examples that follow.

### Expression Vectors.

The murine JAK2 cDNA was cloned into pBluescript II SK vector (Stratagene, La Jolla, Calif.). The JAK2V617F mutation was generated by using site-directed mutagenesis (GeneTailor Site-Directed Mutagenesis System, Invitrogen Carlsbad, Calif.). Following confirmation of the mutation by full-length DNA sequencing, the JAK2V617F and wild-type (WT) cDNA were cloned into the retroviral vector MSCV-IRES-GFP yielding plasmids MSCV-IRES-JAK2-GFPor MSCV-IRES-JAK2V617F-GFP. Cell culture. BaF/3 cells [American Type Culture Collection (ATCC), Manassas, Va.] were grown in RPMI 1640 (Life Technologies, Carlsbad, Calif.) containing 10% fetal bovine serum (FBS; HyClone, Logan, Utah) and 15% WEHI-3B (ATCC)-conditioned medium as a source of murine IL-3 (WEHICM) at 37° C. and 5% CO₂. Cells were transduced by electroporation (300 mV, 20 ms, Bio-Rad Gene Pulser Xcell, Hercules, Calif.) with either MSCV-IRESGFP(MIG)-J AK2 or MIG-JAK2V617F vector. After 48 hours, cells expressing the constructs were selected by fluorescence-activated cell sorting (FACS).

To assess for cytokine hypersensitivity, BaF/3 cells expressing WT and V617F mutant JAK2 were washed with PBS and cultured for 6 days in RPMI 1640 containing 10% FBS in the presence of a WEHI-CM gradient, with parental BaF/3 cells as a control.

For the cell proliferation assay, cells were incubated for 3 hours in CellTiter 96 Solution (Promega, Madison, Wis.) and analyzed using a microplate spectrophotometer (Bio-Rad). For Western blot, BaF/3 cells were washed with PBS and resuspended in RPMI 1640 containing 0.5% bovine serum albumin (BSA; Sigma-Aldrich, St. Louis, Mo.) and incubated for 12 hours. Following incubation, cells were stimulated with F18% FBS and 0.1, 1, 5, and 10 ng/mL IL-3 (PeproTech, Inc., Rocky Hill, N.J.) for 30 minutes. Cells were then washed with ice-cold PBS and 8×10⁵ cells were lysed, loaded on a 4% to 15% Criterion Tris-HCl gel (Bio-Rad), transferred to polyvinylidene fluoride membrane (Millipore, Bedford, Mass.), and blotted with indicated antibodies.

### Retroviral Infection.

For generating retrovirus, Bosc23 cells (ATCC) were grown in DMEM (Life Technologies) containing 10% FBS. Cells were transfected with either MSCV-IRES-GFP(empty vector control), MIG-JAK2, or MIG-JAK2V617F vector using Fugene 6 transfection reagent (Roche, Indianapolis, Ind.). The next day, cells were incubated in fresh medium for an additional 24 hours.

To estimate viral titers, NIH3T3 cells (ATCC) were incubated for 48 hours with graded amounts of viral supernatant added to DMEM containing 10% FBS and 8 Ag/mL polybrene (hexadimethrine bromide, Sigma-Aldrich). Cells were trypsinized (0.05% trypsin-EDTA, Life Technologies), washed in PBS (Life Technologies), and analyzed for green fluorescent protein (GFP) expression by flow cytometry (FACSAria, BD Biosciences, San Diego, Calif.). Titers were estimated by plotting the proportion of GFP-positive cells versus the proportion of retroviral supernatant.

### Bone Marrow Harvest, Infection, and Transplantation.

BALB/c (Charles River Laboratories) donor mice (6-9 weeks old) were primed by retro-orbital injection of 3 mg 5-fluorouracil (5-FU; American Pharmaceutical Partners, Schaumburg, Ill.). After 5 days, bone marrow was harvested from mice by flushing the femur and tibia with medium. Cells were cultured at 5×105 per mL with viral supernatant. After 24 hours, the medium was replaced with fresh retroviral “cocktail” and cells were incubated for additional 24 hours. Next, cells were analyzed for GFP expression by FACS. Viable cells (5×105) were injected retro-orbitally into lethally irradiated (2×450 cGy) syngeneic recipients (MSCV-GFP-empty vector control, n=6; MSCV-IRES-JAK2-GFP, n=12; and MSCV-IRES-JAK2V617F-GFP, n=13).

Blood counts were determined using a Vet ABC animal blood counter (Heska, Fort Collins, Colo.). On day 80 after bone marrow transplantation, four empty vector control mice, four JAK2 mice, and five JAK2V617F mice were euthanized for detailed analysis. For secondary transplant, 1×106 bone marrow cells were injected retroorbitally into sublethally irradiated recipients (1×450 cGy).

### Pathologic Examination

Blood was collected from anesthetized mice by lethal inferior vena cava bleeding and incubated at 5° C. overnight. Serum was harvested by centrifugation at 4,600 rpm for 10 minutes. Organs were dissected and WBC from spleen and bone marrow were harvested by passing through a 70-Am nylon cell strainer (BD Biosciences, Bedford, Mass.) followed by red cell lysis. For colony-forming assays, 1×105 leukocytes from the spleen of mice were plated with 1 mL medium in methycellulose, without cytokines (Methocult M3234), with cytokines without Epo (Methocult GF M3534), and with cytokines plus Epo (Methocult GF M3434, StemCell, Vancouver, British Columbia, Canada) and incubated at 37° C. and 5% CO₂ for 20 days.

### Histologic and Immunohistochemical Analysis of Murine Tissue

Samples from liver, spleen, heart, humerus, brain, and lung were fixed in 3.5% paraformaldehyde (Protocol, Kalamazoo, Mich.) and embedded in paraffin. Brains were preserved by equilibration in increasing concentrations of sucrose (10%, 20%, and 30%). Sections of 5 μm were stained with H&E. Decalcified sections of humerus were stained with H&E and reticulin stain (American Master Tech Scientific, Inc. Lodi, Calif.).

### Flow Cytometric Analysis

White cells from bone marrow and spleen were resuspended in staining buffer (BD Biosciences). Aliquots of 1×106 cells were stained for 20 minutes with conjugated monoclonal antibodies. Cells were washed in staining buffer and analyzed by FACS. For FACS analysis of intracellular JAK2 phosphorylation, peripheral blood of one JAK2V617F mouse and three normal BALB/c mice were collected. WBC from normal BALB/c mice were pooled together. Cells were split into three aliquots and resuspended in RPMI 1640 (Life Technologies) containing 0.5% BSA and incubated for 15 hours. Following incubation, one aliquot was stimulated with 10 ng/mL IL-3 plus 10 ng/mL granulocyte colonystimulating factor (G-CSF; StemCell) and second aliquot was stimulated with 10 ng/mL IL3, 10 ng/mL G-CSF, and 10 Amol/L AG-490 (Calbiochem) for 60 minutes and third aliquot was left unstimulated. Cells (1×105) were resuspended in 50 AL staining buffer (1% BSA and 0.1% sodium azide in PBS) and permeabilized using Fix&Perm cell permeabilization kit (Caltag Laboratories, Burlingame, Calif. and An der Grub, Vienna, Austria). The granulocyte population was selected by forward and side scatter

### Southern Blot Analysis.

Genomic DNA was isolated from leukocytes extracted from the spleen of mice using DNeasy Tissue kit (Qiagen, Valencia, Calif.). For Southern blot analysis, 10 to 15 Ag genomic DNA was digested with BglII, resolved on a 1.5% agarose gel, and transferred to Hybond-N+ membrane. The blots were hybridized with a 32P-labeled GFP probe and exposed to autoradiography film.

### Quantification of Serum Cytokine Levels.

For measurement of Epo levels, the Quantikine mouse Epo kit (R&D Systems, Minneapolis, Minn.) was used according to the manufacturer's instructions. Serum levels of IL-2, IL-6, tumor necrosis factor (TNF)-a, GM-CSF, and G-CSF were measured using a bead-based immunoflourescence assay (Luminex, Inc. Austin Tex.), using Millipore multiscreen 96-well filter plates. Cytokine analysis kits (LINCOplex kits) were purchased from Linco Research, Inc. (St. Charles, Mo.). Assays were run in duplicate according to the manufacturer's instructions. A fourvariable regression formula was used to calculate the sample concentrations from the standard curves.

### Statistics.

The SPSS statistics package was used for all statistical analyses. Continuous variables were compared by pairwise t test for two independent samples.

### Example 2

### JAK2V617F Induces IL-3 Hypersensitivity in BaF/3 Cells

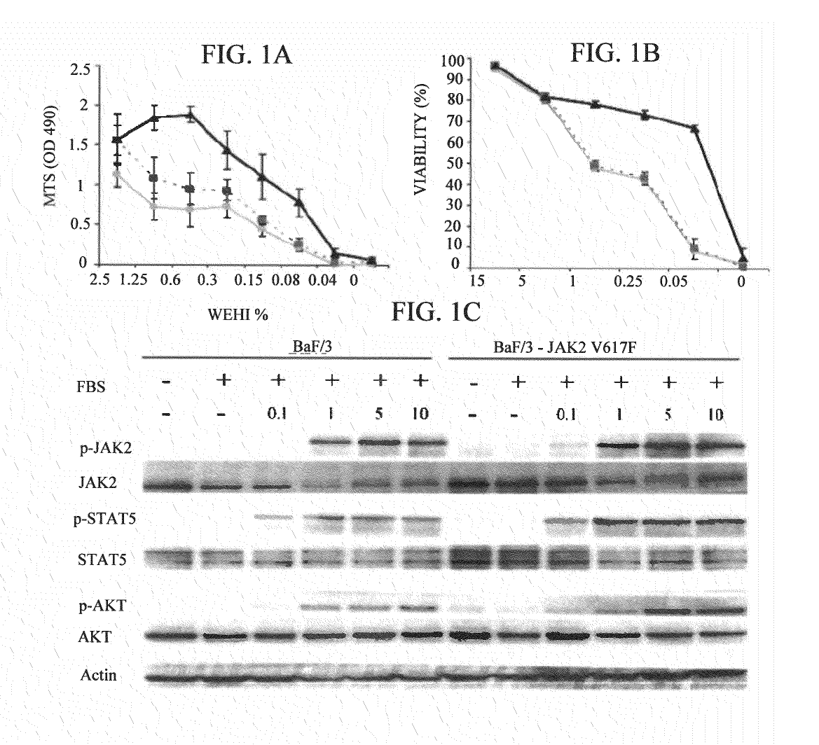

Murine JAK2V617F and WT JAK2 (JAK2WT) was expressed in the murine IL-3-dependent cell line BaF/3. The cells were cultured for 6 days in medium containing graded concentrations of WEHI-CM. Cells expressing JAK2V617F showed significantly increased proliferation (FIG. 1A) and viability (FIG. 1B) in low concentrations of IL-3 compared with cells expressing JAK2WT or parental BaF/3 cells. No difference was observed in the absence of IL-3, indicating that JAK2V617F induces cytokine hypersensitivity but not complete cytokine independence, consistent with the observation that primary PV progenitor cells are hypersensitive to cytokines but not cytokine independent. The tyrosine phosphorylation of JAK2 was monitored by immunoblot analysis after serum starvation for 12 hours and stimulation with increasing IL-3 concentrations for 30 minutes (FIG. 1C). BaF/3 cells expressing JAK2V617F showed only a slight increase of JAK2 phosphorylation compared with parental BaF/3 cells. A more pronounced difference was detected in the phosphorylation of the JAK2 substrate signal transducer and activator of transcription 5 (STAT5) as well as Akt, a downstream effector of EpoR signaling. Consistent with the lack of cytokine-independent growth, no JAK2 autophosphorylation was observed in BaF/3 cells expressing JAK2V617F in the absence of cytokine stimulation.

### Example 3

### JAK2V617F Induces Trilineage MPD in Mice

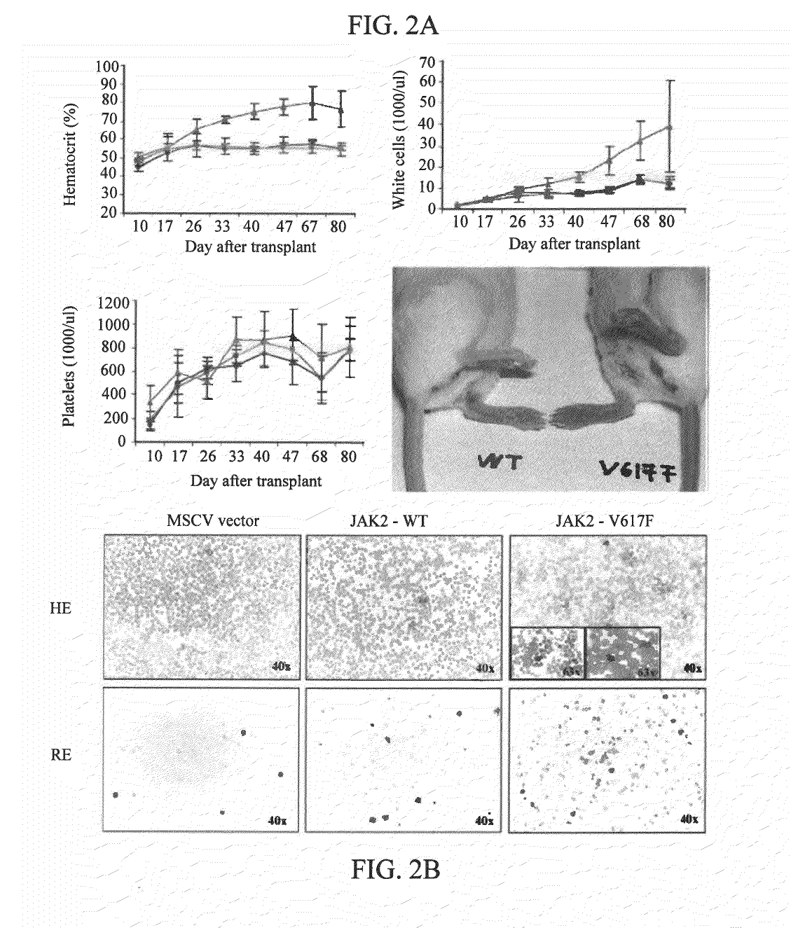

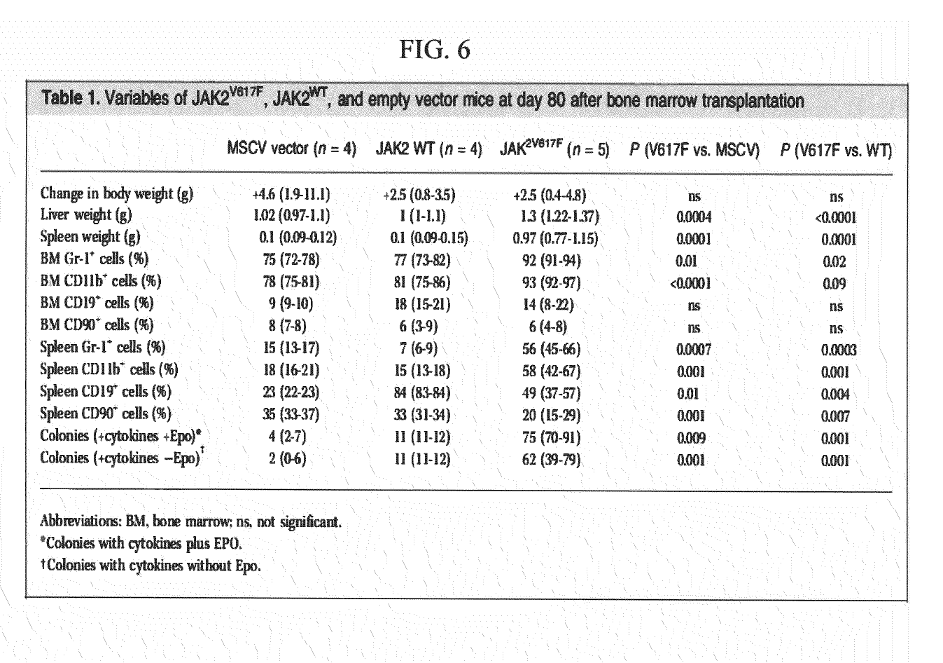

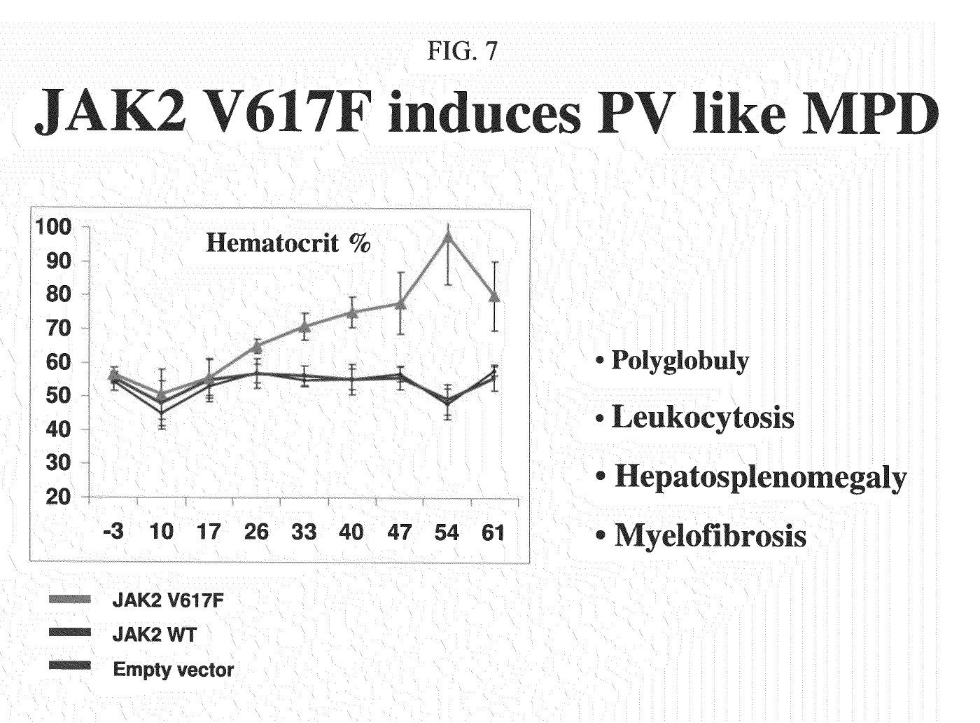

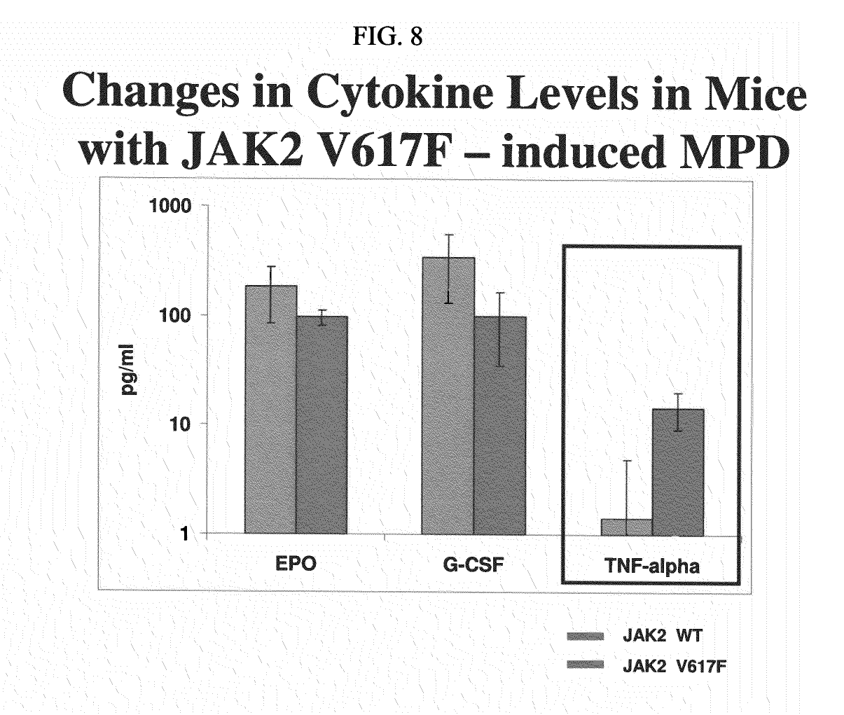

To examine the oncogenic potential of JAK2V617F in vivo, one marrow from 5-FU-treated male BALB/c mice was infected with MSCV-IRES-GFP(MIG), MIG-JAK2WT, or MIG-JAK2V617F. FACS analysis for GFP expression of infected bone marrow after two rounds of infection showed a low rate of GFP-positive cells for all three constructs (<1%). Equal numbers of viable cells were injected into lethally irradiated syngeneic recipients. Recipients of marrow infected with JAK2WT and empty vector maintained normal blood counts during the entire observation period (166 days). In contrast, recipients of JAK2V617F-infected marrow exhibited erythrocytosis 26 days after transplantation (FIG. 2A). Erythrocytosis was defined as hematocrit >58% and hemoglobin >18 g/dL. All blood variables are based on reference range peripheral blood variables of healthy untransplanted BALB/c mice. Maximum hematocrit values varied between 57% and 89%, with a median of 83%, and maximum hemoglobin levels varied between 18 g/dL and 28 g/dL, with a median of 24 g/dL. Mice with erythrocytosis developed skin plethora (FIG. 2A). Leukocytosis (defined as white cell counts >18,000/AL) developed in 12 of 13 mice. In contrast to red cell variables, maximal white cell counts were quite variable, ranging from normal to 120,000 per AL, with a predominance of granulocytes. In two JAK2V617F mice, slightly elevated platelet counts were observed over a period of 4 weeks, but median platelet counts in the cohort as a whole were not different from controls (FIG. 2A). Thrombocytosis was defined as platelets >850,000/AL. Thus, most JAK2V617F mice developed a disease that closely resembled PV. However, none of the mice died from their disease and did not exhibit significant symptoms, such as weight loss (Table 1, see FIG. 6).

### Example 4

### JAK2V617F Induces Extramedullary Hematopoiesis

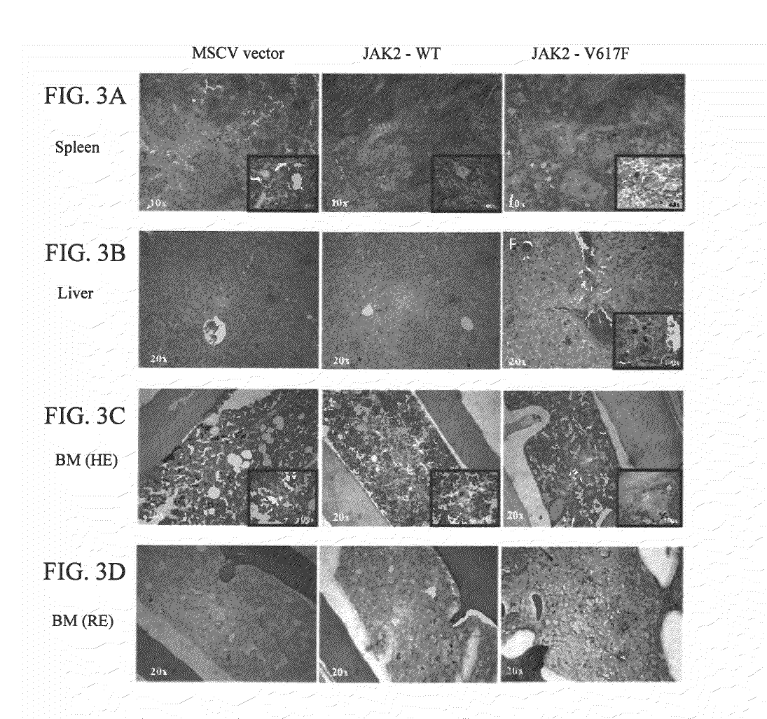

The peripheral blood smears of JAK2WT and empty vector mice were morphologically normal. In contrast, JAK2V617F mice showed prominent abnormalities in the red cells, including nucleated red cells, anisocytosis, spherocytes, and reticulocytosis (FIG. 2B). White cells were predominantly mature and morphologically normal neutrophils. Randomly selected JAK2V617F, JAK2WT, and empty vector mice were sacrificed for detailed examination at day 80 posttransplantation, whereas observation of the remaining mice continued (latest follow-up, day 166). Necropsy revealed mild hepatomegaly and significant splenomegaly in all recipients of JAK2V617F-infected marrow (Table 1). All other organs appeared macroscopically normal. Histopathology of the spleen showed normal white pulp with minimal extramedullary hematopoiesis in JAK2WT and empty vector mice. Some megakaryocytes were present but were morphologically unremarkable (FIG. 3A). In contrast, the white pulp of JAK2V617F mice showed lymphoid hyperplasia and extramedullary hematopoiesis. The red pulp was expanded, with extensive extramedullary hematopoiesis and effacement of splenic architecture. Extramedullary erythropoiesis and myelopoiesis were normal, except for the presence of dysplastic megakaryocytes with increased size and large, lobular nuclei and apoptotic features, such as condensed pyknotic nuclei (FIG. 3A). The livers of JAK2V617F mice also displayed infiltration by hematopoietic cells with occasional atypical megakaryocytes, although not to the degree observed in the spleens (FIG. 3B). Livers of JAK2WT and empty vector mice showed no abnormalities (FIG. 3B). The bone marrow histology of mice transplanted with JAK2WT or empty vector-infected cells was normal with trilineage hematopoiesis, a myeloid to erythroid ratio of 1:1 to 2:1 and morphologically normal megakaryocytes (FIG. 3C). In contrast, JAK2V617F mutant mice showed hypercellular marrow with rightshifted granulocytic hyperplasia and megakaryocytic hyperplasia (FIG. 3C). Megakaryocytes appeared in clusters, most prominently around sinuses and again exhibited dysplastic features, such as increased cell and nuclear size and abnormal nuclear lobation, and apoptotic features. Unexpectedly, erythroid cells were overall markedly reduced. A reticulin fiber stain showed no increase in reticulin fibers in JAK2WT and empty vector control mice but moderate to severe reticulin fibrosis in JAK2V617F mice (FIG. 3D). Altogether, the histologic findings are consistent with a trilineage MPD resembling PV. However, the finding of reduced erythropoiesis in the marrow suggests that on day 80 the disease had evolved to a stage comparable with the ‘spent phase’ of PV with postpolycythemic myelofibrosis. Thromboembolic events are common in patients with PV and ET. Therefore sections of heart and brain were evaluated for signs of embolic infarctions or thrombosis. Microscopically, these tissues appeared normal, without obvious differences between JAK2V617F mice and controls. This is consistent with the lack of clinical signs and symptoms in JAK2V617F mice.

### Example 5

### Constitutive Jak2 Phosphorylation in Murine Leukocytes Expressing JAK 2V617F

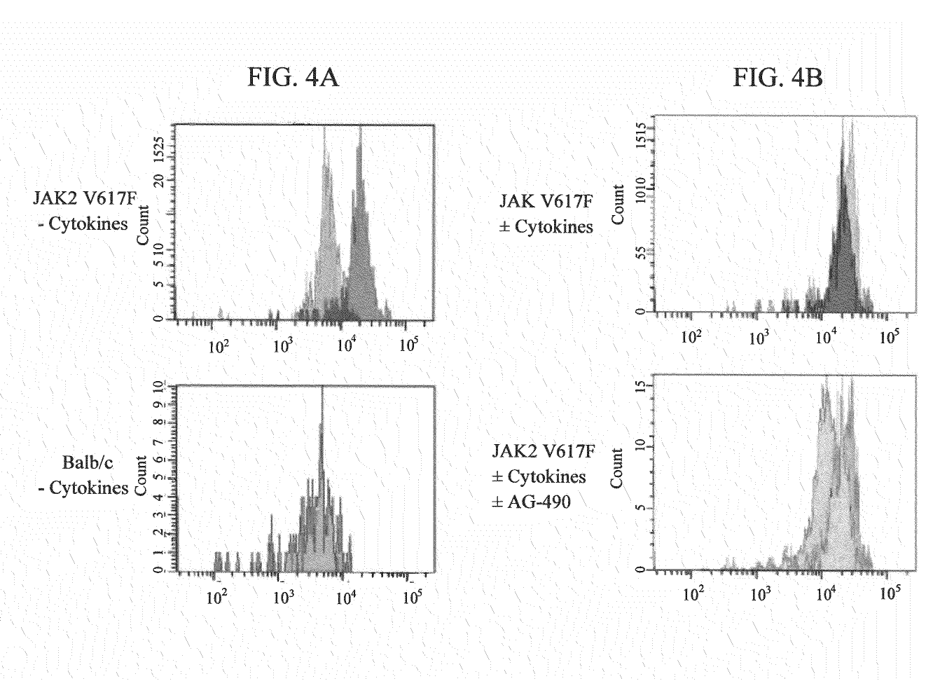

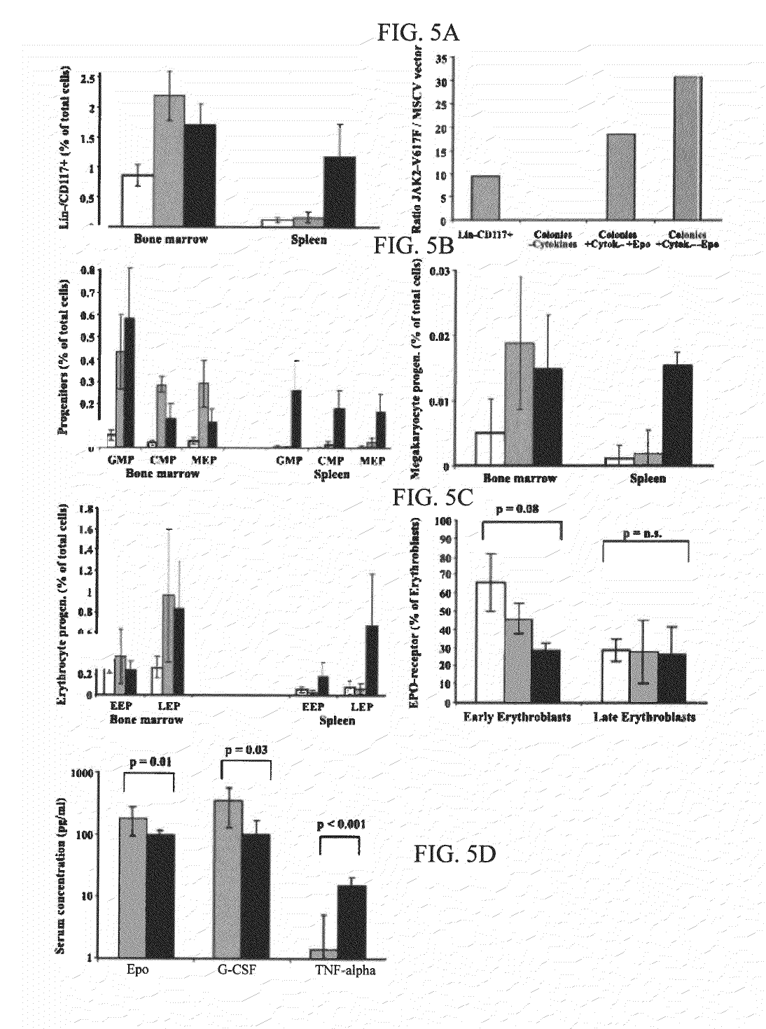

To test if the mutant JAK2V617F kinase in murine leukocytes is constitutively active, peripheral blood granulocytes was analyzed by FACS for phosphorylated JAK2. Peripheral blood leukocytes of a JAK2V617F mouse and normal control BALB/c mice were cultured in cytokine-free medium for 15 hours. Subsequently, aliquots of cells were stimulated in the presence and absence of IL-3 and G-CSF and/or 10 Amol/L AG490, a JAK2 inhibitor. The JAK2V617F mouse was leukemic with a white cell count of 39,000 cells/AL and 41% GFP-positive peripheral WBC. FACS analysis showed pronounced JAK2 phosphorylation in cytokine unstimulated GFP-positive compared with GFP-negative peripheral blood granulocytes of the JAK2V617F mouse and compared with granulocytes of normal BALB/c mice (FIG. 4A). After cytokine stimulation for 60 minutes, the GFP positive peripheral blood granulocytes of the JAK2V617F mouse showed a further increase in JAK2 phosphorylation (FIG. 4B, top). Treatment with AG490 reduced phosphorylated JAK2 levels below baseline, consistent with constitutive JAK2 phosphorylation in the leukemic cells (FIG. 4B, bottom).

### Example 6

### JAK2V617F-Induced MPD is Oligoclonal

DNA extracted from the spleen of four JAK2V617F mice and one JAK2WT mouse was probed with GFP to determine the numbers of retroviral integrations/clones. Two JAK2V617F mice showed one band (lanes 2 and 4) and two mice showed two bands (lane 3), consistent with a monoclonal or biclonal leukemic population. No bands were distinguishable in JAK2WT mice (lane 1). The low number of retroviral integration sites is in line with the low infection rate of transplanted bone marrow. JAK2V617F induces expansion of hematopoietic stem/progenitor cells. To define more precisely the lineage and differentiation stages of the hematopoietic cells involved in the JAK2V617F-induced MPD, multicolor FACS analysis of bone marrow and spleen cells and immunohistochemistry on tissue sections was done. In the bone marrow, the proportion of mature myeloid cells, characterized by expression of Gr-1 and CD11b, was consistently increased in JAK2V617F mice compared with controls (Table 1). As expected from light microscopy, there were few lymphoid cells (CD19+ or CD90+), with little difference between the groups. FACS of splenic cells from JAK2V617F mice revealed >50% of cells expressing myeloid markers (Table 1), consistent with myeloperoxidase positivity by immunohistochemistry. The proportion of cells with a B-cell phenotype (CD19+) was increased in both JAK2WT and JAK2V617F mice compared with empty vector mice, consistent with the histologic finding of lymphoid hyperplasia in the white pulp and confirmed by immunohistochemistry using the B220 monoclonal antibody. The proportion of bone marrow cells with a T-cell phenotype (CD90+) was comparable between the three groups, but slightly reduced in the spleen of JAK2V617F mice, reflecting the relative increase in myeloid cells. The fact that JAK2V617F is found in PV, ET, and IMF suggests that the mutation may be acquired by a multipotent stem cell, with additional factors determining the disease phenotype. Consistent with this notion, recent reports have suggested that JAK2V617F—positive ET is closely related to PV, with extrinsic and possibly genetic factors modulating the penetrance of the PV phenotype. The BALB/c mice used in our experiments express only low levels of Sca-1; therefore, identification of stem cells based on the combination of CD117 (KIT) and Sca-1 expression was not possible. However, using a combination of lineage negativity and CD117 positivity, a cell population can be identified that contains stem and progenitor cells. JAK2V617F mice had a significantly higher percentage of lineage-negative (Lin−)/CD117+ cells in the bone marrow and spleen compared with mice transplanted with marrow infected with vector control (FIG. 5A, left). Unexpectedly, the Lin−/CD117+ population was also expanded in the marrow of mice transplanted with JAK2WT-infected marrow. A much more pronounced expansion of Lin−/CD117+ cells in the spleens of JAK2V617F mice relative to empty vector and JAK2WT was observed. The small proportion of stem and progenitor cells in the spleens of the JAK2WT mice (˜0.2%) and the vector control mice (˜0.1%) is consistent with the presence of minimal extramedullary hematopoiesis in spleens of normal mice. The data shows that JAK2V617F leads to a significant expansion of the stem and progenitor cell compartment in the spleen and the bone marrow. The relatively discrete expansion in the marrow may reflect the fact that at the time of the histology the disease had already evolved to ‘spent phase’.

To investigate whether the clonogenic activity of splenic cells was correlated with the relative proportion of stem and progenitor cells (Lini/CD117+ cells) in the spleens of JAK2V617F mice compared with controls, colony formation was assessed in semisolid medium. Equal numbers of leukocytes isolated from the spleens were plated in methylcellulose with and without cytokines (IL-3, IL-6, and Scf) and with and without Epo. After 20 days in culture, colonies were counted (Table 1). No growth was observed in any of the groups in the absence of cytokines. In the presence of cytokines plus Epo, a 19-fold increase of colonies was observed in cultures from JAK2V617F mice compared with empty vector mice, which is significantly higher than the 10-fold increase of Lin−/CD117+ cells in the JAK2WT mice compared with empty vector mice, suggesting increased clonogenic activity per Lin−/CD117+ cells in optimal growth factor concentrations (FIG. 5A, right). With cytokines but without Epo, a more pronounced difference in colony formation between JAK2V617F mice and empty vector mice was observed, consistent with increased clonogenic potential of JAK2V617F cells under suboptimal growth factor conditions.

To determine how the relative proportions of the earliest committed progenitor cells are affected by JAK2V617F, Lin−/CD117+ cells according to Fcg receptor and CD34 expression were analyzed by FACS, this allows separating the cell population into common myeloid progenitors (CMP), granulocyte-monocyte progenitors (GMP), and megakaryocyte-erythrocyte progenitors (MEP). The proportion of the three different progenitor populations (CMP, GMP, and MEP) to each other was similar to published data in C57BL mice, which express Sca-1 on their hematopoietic stem cells. In the bone marrow, both JAK2WT and JAK2V617F mice showed a relative increase of GMP, CMP, and MEP (FIG. 5B, left), in line with the overall expansion of Lin−/CD117+ cells in the marrow. The spleens of JAK2V617F mice contained significantly increased CMP, GMP, and MEP compared with the empty vector and JAK2WT control groups. The relative proportions of GMP, CMP, and MEP were similar in the bone marrow and spleens of JAK2V617F mice. Comparison with normal bone marrow suggests that JAK2V617F expression does not seem to favor expansion of one particular progenitor cell population, as the relative expansion of GMP, CMP, and MEP is similar. This is in contrast to recent findings in PV patients with high white cell counts who show a significant increase of the CMP population in the peripheral blood. The JAK2V617F mutation causes expansion of late progenitors. As erythrocytosis is not commonly observed in murine MPD models, Epo levels were determined to exclude secondary erythrocytosis. JAK2V617F mice had low Epo levels compared with JAK2WT mice (95 pg/mL compared with 182 pg/mL; P=0.017; FIG. 5D). To determine more precisely at which differentiation stage the expansion of the red cell compartment occurred, early erythroid progenitor (EEP) and late erythroid progenitor (LEP) was assessed by FACS. Gates were set to include Lin−/+ cells, with EEP defined as CD71low and LEPas CD71high. In contrast to vector controls, JAK2V617F but also JAK2WT mice showed a predominance of LEP over EEP in their bone marrow (FIG. 5C, left). Similar results were seen in the spleens of JAK2V617F mice. These data suggest that JAK2V617F predominantly induces expansion of the LEPcell compartment. The preferential expansion of the erythrocyte compartment may be due to increased expression of the EpoR on the cells. Early erythroblast cells were gated as lineage positive (Lin+), CD71high, and Ter-119med/high, and late erythroblast cells were gated as Lin+, CD71low/negative, and Ter-119high. For the late erythroblast population, EpoR was not different between the three groups of mice (FIG. 5C, right). In contrast, a reduction of EpoR expression was observed on early erythroblasts of JAK2V617F mice that reached borderline significance (P=0.08). The proportion of megakaryocyte progenitors was also analyzed, identified as Lin−/CD117+/CD9+/Fcg receptor low/CD41+. The JAK2V617F mutation causes expansion of late progenitors. As erythrocytosis is not commonly observed in murine MPD models, Epo levels were determined to exclude secondary erythrocytosis. JAK2V617F mice had low Epo levels compared with JAK2WT mice (95 pg/mL compared with 182 pg/mL; P=0.017; FIG. 5D). To determine more precisely at which differentiation stage the expansion of the red cell compartment occurred, early erythroid progenitor (EEP) and late erythroid progenitor (LEP) were assessed by FACS. Gates were set to include Lin−/CD117+ cells, with EEP defined as CD71low and LEPas CD71high. In contrast to vector controls, JAK2V617F but also JAK2WT mice showed a predominance of LEPover EEP in their bone marrow (FIG. 5C, left). Similar results were seen in the spleens of JAK2V617F mice. These data suggest that JAK2V617F predominantly induces expansion of the LEP cell compartment. The preferential expansion of the erythrocyte compartment may be due to increased expression of the EpoR on the cells. Early erythroblast cells were gated as lineagepositive (Lin+), CD71high, and Ter-119med/high, and late erythroblast cells were gated as Lin+, CD71low/negative, and Ter-119high. For the late erythroblast population, EpoR was not different between the three groups of mice (FIG. 5C, right). In contrast, a reduction of EpoR expression was observed on early erythroblasts of JAK2V617F mice that reached borderline significance (P=0.08). The proportion of megakaryocyte progenitors, identified as Lin_/CD117+/CD9+/Fcg receptorlow/CD41+ was also analysed. The bone marrow and spleens of JAK2V617F mice showed a 3- and 15-fold relative increase of the megakaryocyte progenitor population compared with empty vector mice (FIG. 5B, right). As seen earlier with LEP, the bone marrow of JAK2WT mice also showed a relative increase of the megakaryocyte progenitor population compared with empty vector controls.

### Example 7

### Increased Levels of TNF-Alpha and Reduced Levels of G-CSF in Jak2V617F Mice

Cells from patients with PV are hypersensitive to various cytokines in addition to Epo. Serum levels of IL-2 and IL-6 were raised during progression of patients with ET and PV to myelofibrosis. In addition to decreased Epo serum levels other cytokines involved in regulation of hematopoiesis could be affected by JAK2V617F expression. No differences were found in serum levels of IL-2, IL-6, and GM-CSF between JAK2WT and JAK2V617F mice, whereas average serum levels of G-CSF were significantly lower (99 pg/mL compared with 343 pg/mL; P=0.037) in JAK2V617F mice compared with JAK2WT mice (FIG. 5D). In contrast, average serum levels of TNF-alpha were significantly higher in JAK2V617F mice (15 pg/mL compared with 1 pg/mL; P<0.001).

### Example 8

### Long-Term Outcome of JAK2V617F Mice and Secondary Transplants

A cohort of six JAK2V617F mice was followed beyond day 80 for up to 166 days. None of these mice showed clinical symptoms of disease. Peripheral blood counts were extremely variable. FACS analysis for GFP expression detected only low numbers of GFP-positive cells (4-7.7%) in three of six mice; two mice were negative for GFP and one mouse with a WBC of 114,000/AL had 50.6% of GFP-positive cells. These data are consistent with reestablishment of normal nonleukemic hematopoiesis in the peripheral blood of most of the mice. Secondary transplants into sublethally irradiated mice were also done. Two of eight mice developed transient MPD characterized by high hematocrit (>58%) over 30 days, but subsequently hemoglobin levels normalized. Both mice had received marrow from the same donor mouse. All other mice have maintained normal peripheral blood counts up to 115 days after transplantation.

### Example 9

### Effects of JAK2 on TNF-Alpha Knockout Mice

This example provides exemplary procedures for determining the effect of JAK2 on the production of TNF-alpha.

### Transplantation Groups

Group1: 5-FU stimulated TNF-alpha wild type bone marrow (normal bone marrow, able to produce TNF-alpha), retroviral infected for three days in culture (with cytokines) with MSCV-GFP-empty vector control, JAK2-WT or JAK2-V617F, was transplanted into lethal radiated TNF-alpha wild type mice.
Group2: 5-FU stimulated TNF-alpha knock out (KO) bone marrow (bone marrow unable to produce TNF-alpha), retroviral infected for three days in culture (with cytokines) with MSCV-GFP-empty vector control, JAK2-WT or JAK2-V617F, was transplanted into lethal radiated TNF-alpha wild type mice.
Group3: 5-FU stimulated TNF-alpha knock out bone marrow, retroviral infected for three days in culture (plus cytokines) with JAK2-V617F, was transplanted into radiated TNF-alpha knock out mice.

### Results of Transplantation Trials

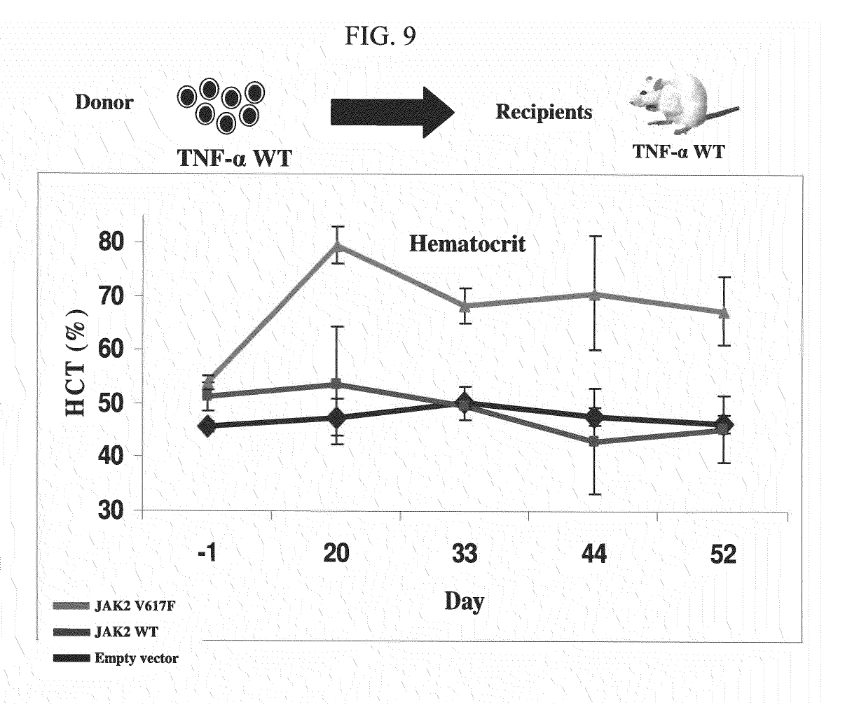

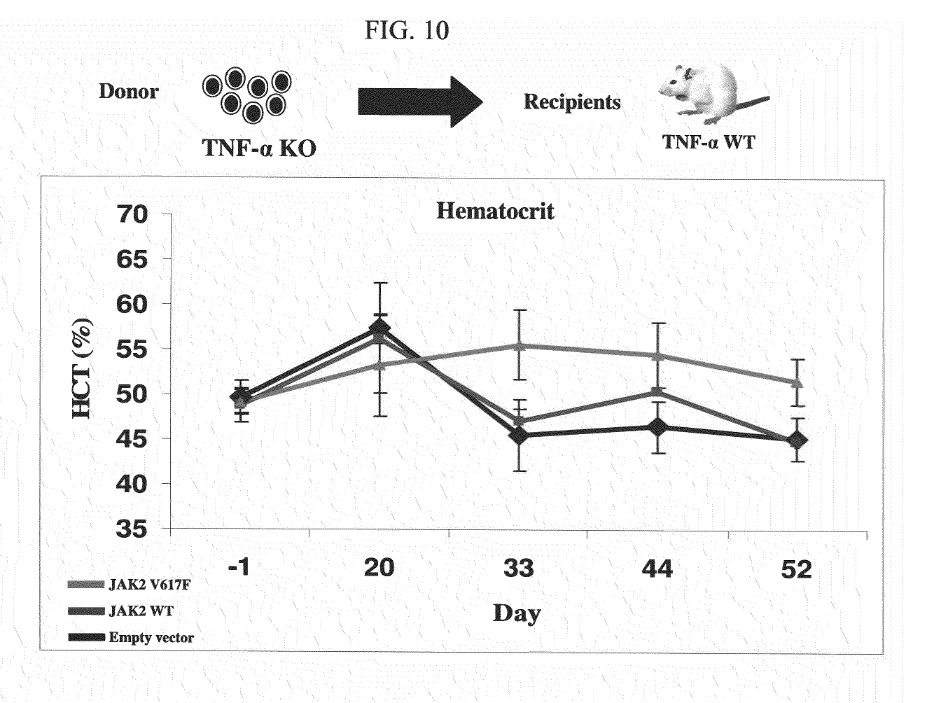

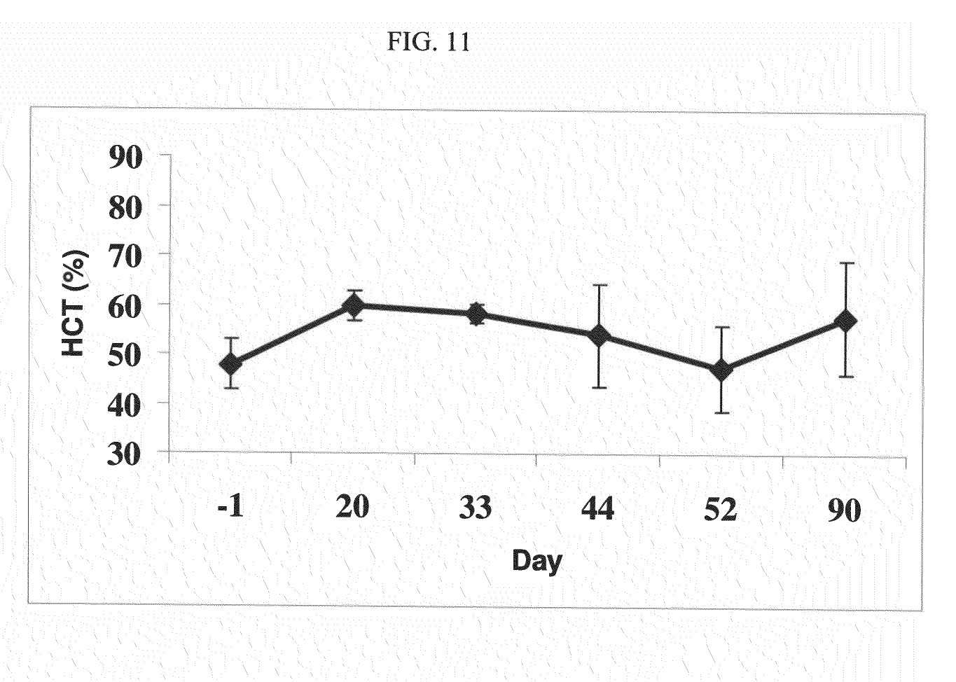

Group1: This transplant group demonstrated that murine TNF-alpha wild type bone marrow infected with JAK2-V617F retrovirus induced a PV like disease in TNF-alpha wild type mice (see FIG. 9).
Group2: It was very surprising to see a complete absence of PV like disease in mice transplanted with TNF-alpha knock out bone marrow cells carrying JAK2-V617F mutation (see FIG. 10). These JAK2-V617F mutant mice have complete normal peripheral blood counts compare to wild type and empty vector control mice. This result demonstrated that TNF-alpha was required for the induction of PV in presence of the PV inducing JAK2 mutation JAK2-V617F.
Group3: These significant TNF-alpha/JAK2-V617F findings were reproduced in TNF-alpha knock out recipient mice (a complete TNF-alpha deficient situation, the donor bone marrow and the recipient mice are TNF-alpha knock out and unable to produce TNF-alpha) (see FIG. 11). This experiment demonstrates, that absence of JAK2-V617F induced disease in Group-2 is not due to a possible host-versus-graft reaction, where recipient mice reject transplanted bone marrow (because recipient TNF-alpha mice (B6; 129S6-Tnf^{tm1Gk1}/J) are a different strain of mice as TNF-alpha wild type donor marrow (B6; 129SF1/J)).

### Necropsy:

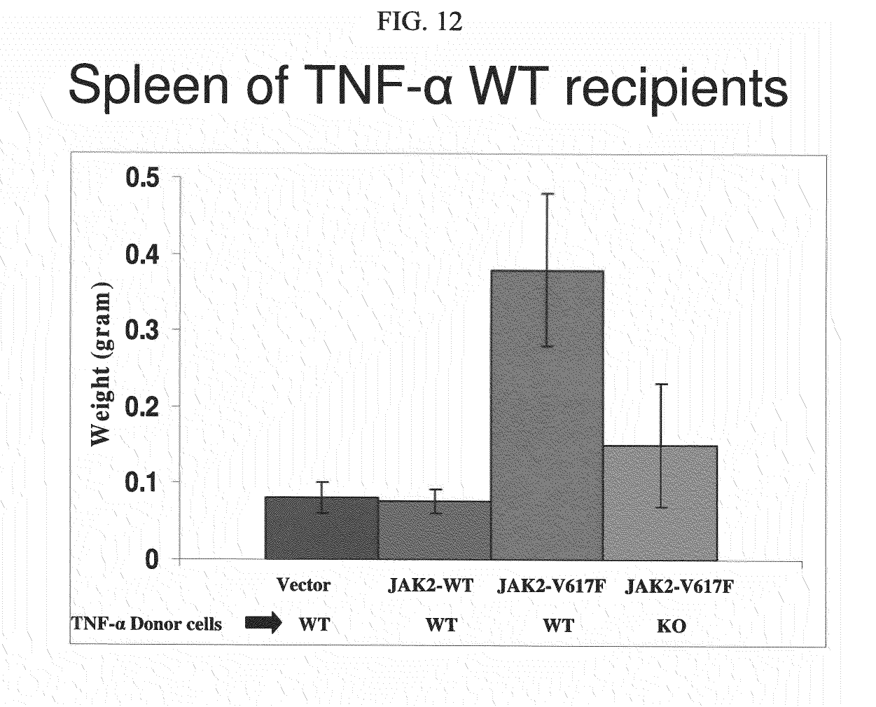

Necropsy revealed in recipient mice transplanted with JAK2-V617F infected TNF-alpha wild type bone marrow, with clear picture of PV like disease in peripheral blood counts (see FIG. 9), significant splenomegaly (see FIG. 12). In contrast mice transplanted with JAK2-V617F infected TNF-alpha knock out bone marrow, with normal peripheral blood counts (see FIG. 10), had no statistic significant increase in spleen weight (see FIG. 12). Mice transplanted with MSCV-GFP empty vector or JAK2-WT construct had normal spleen (see FIG. 12).

### Histopathology:

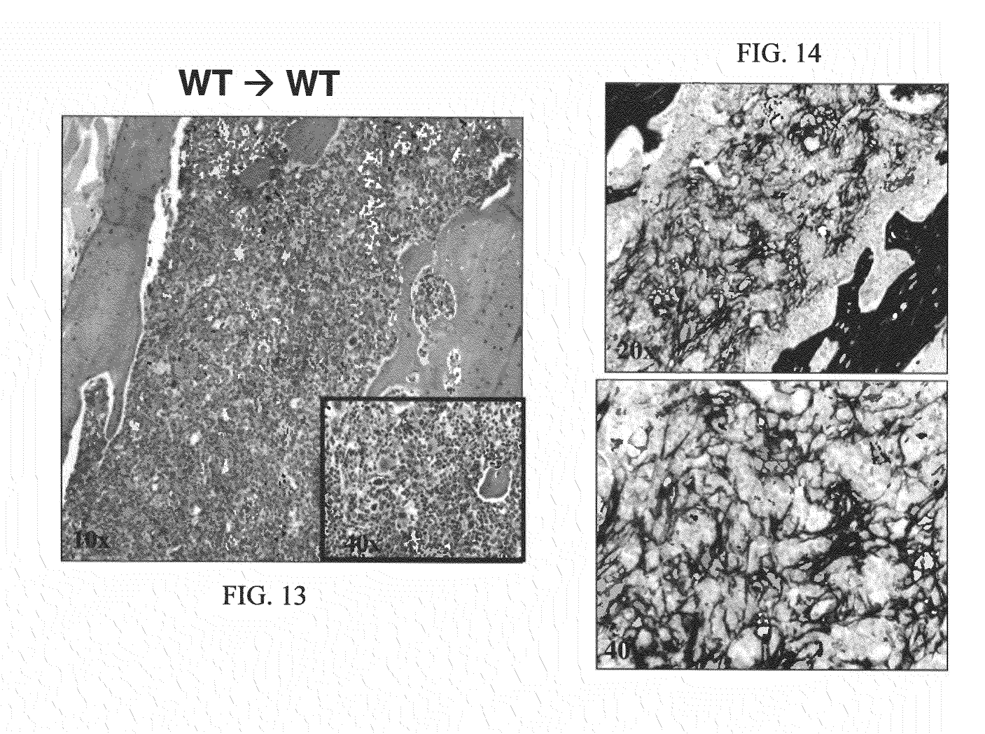

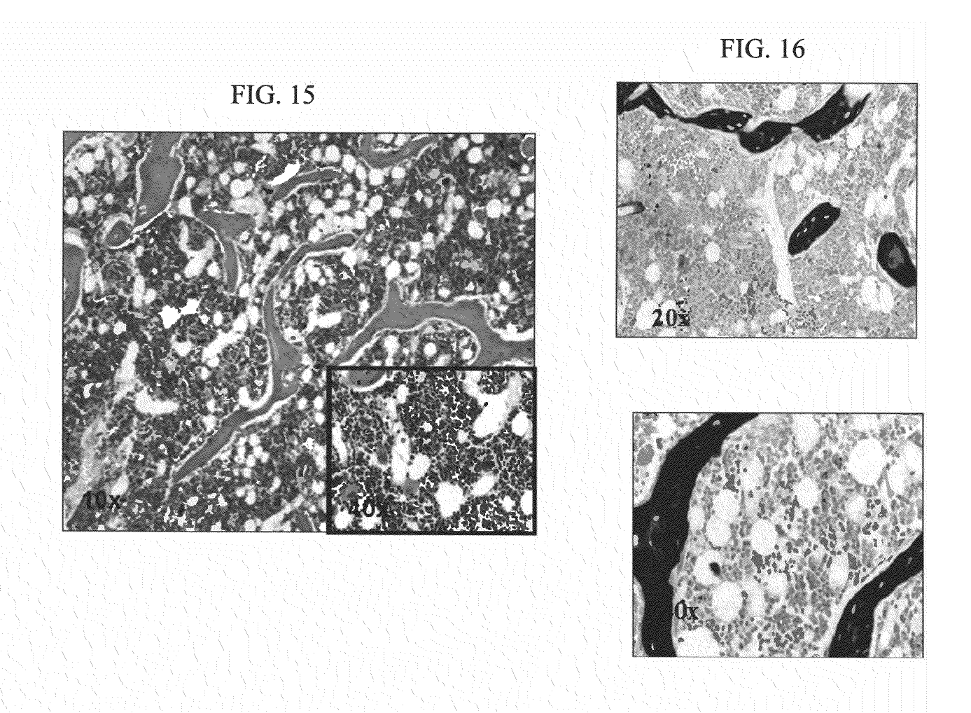

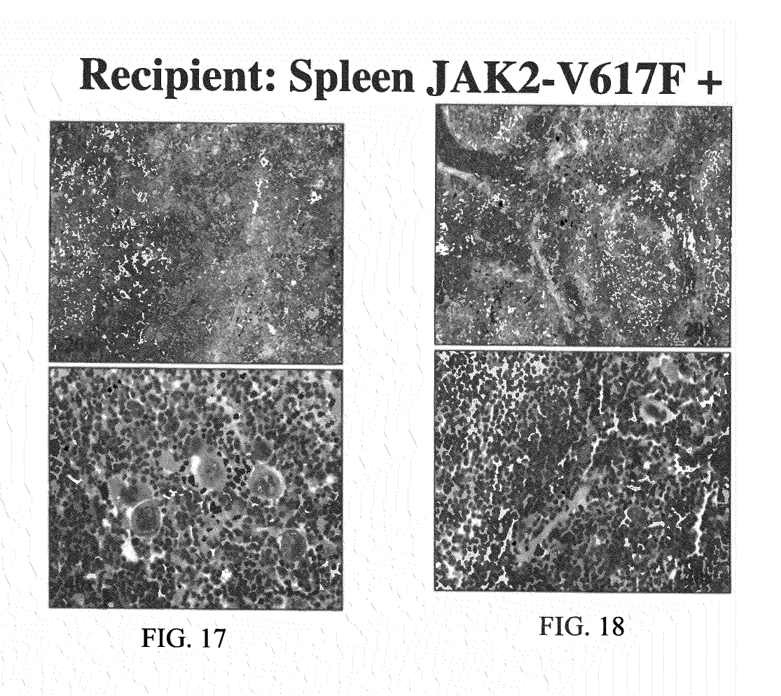

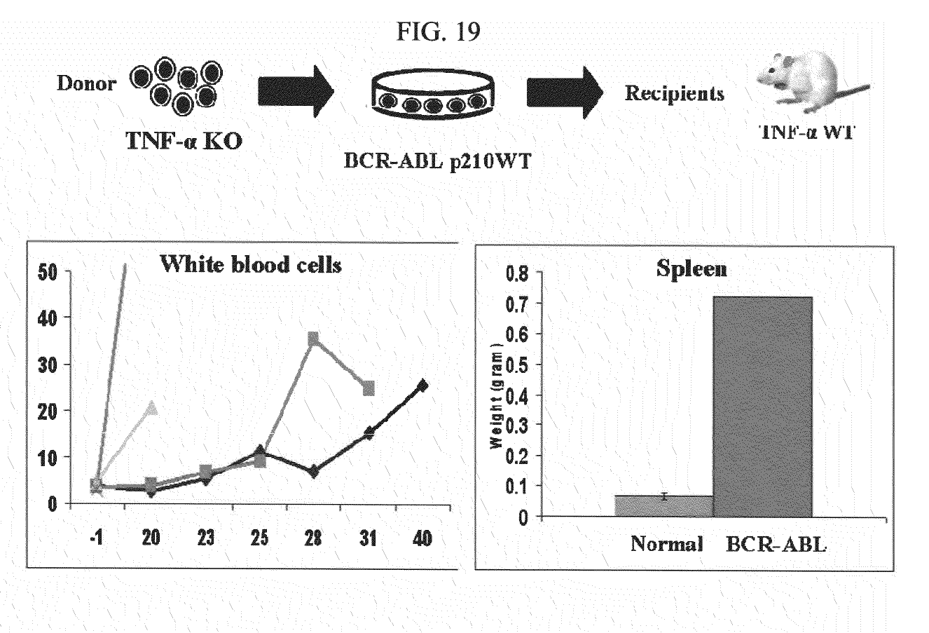

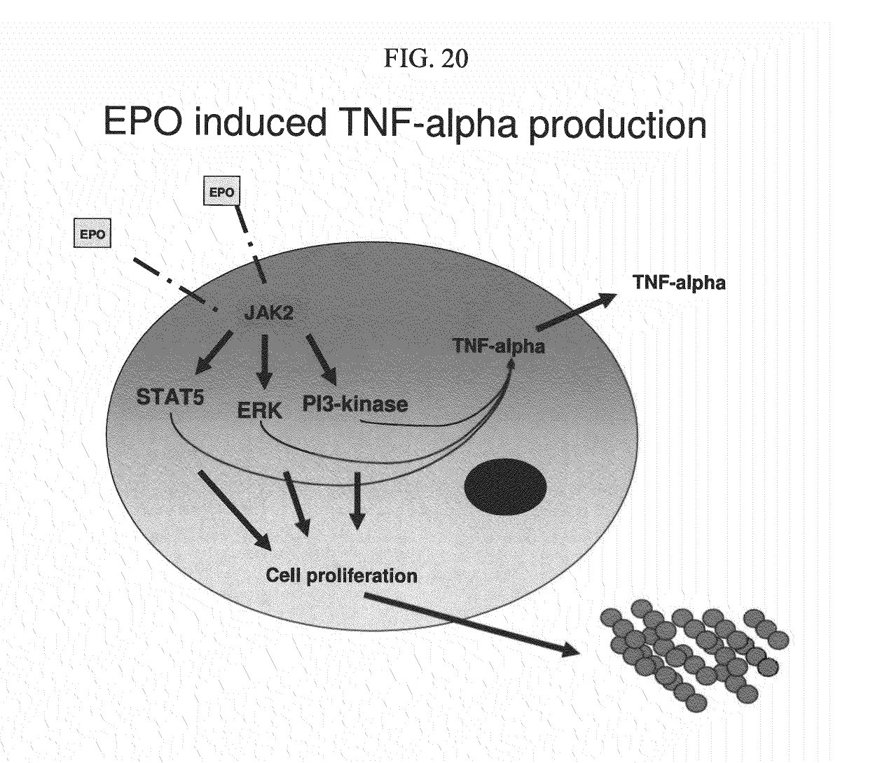

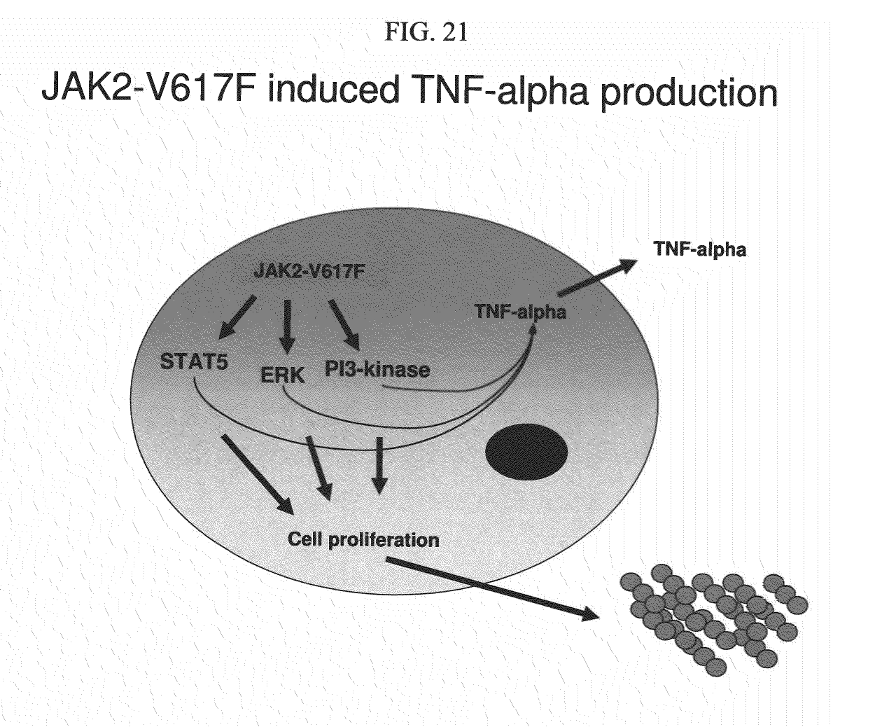

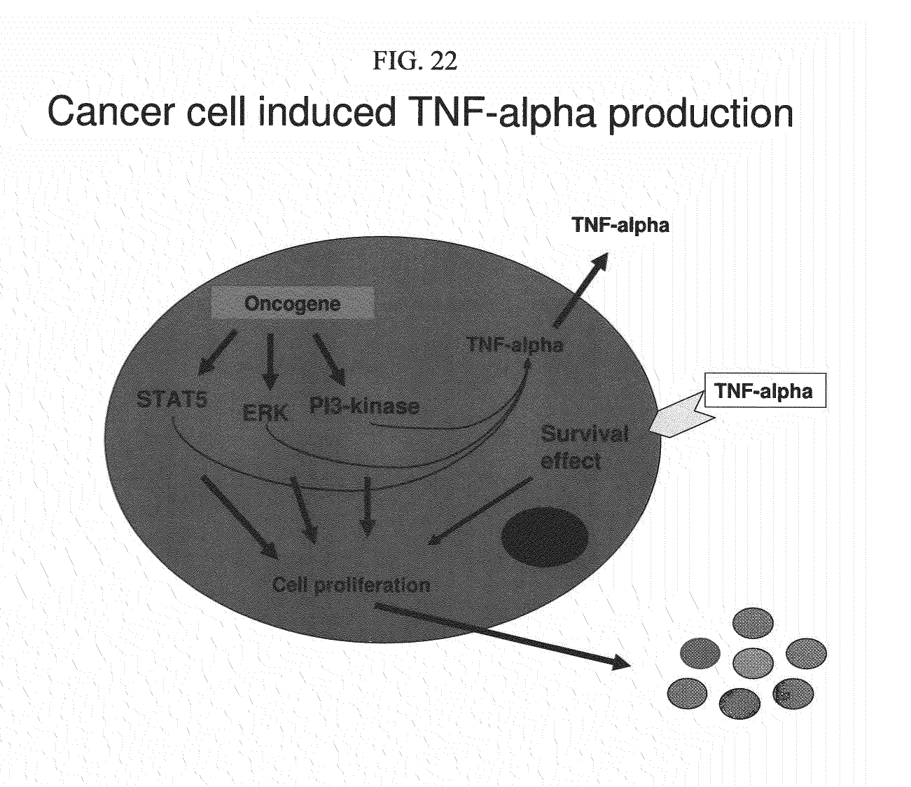

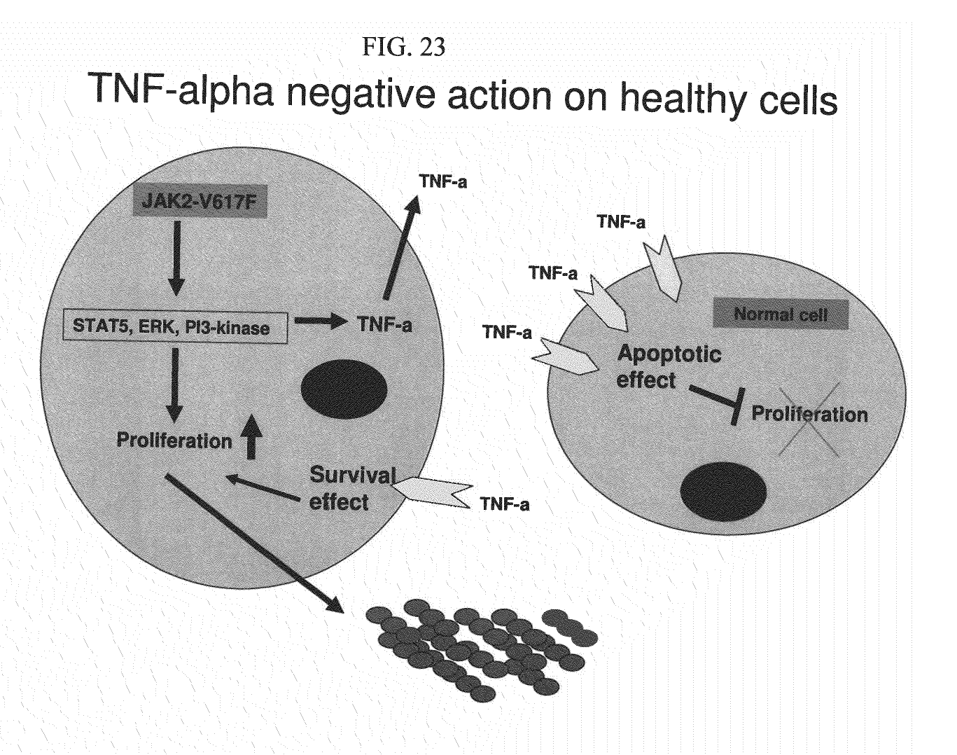

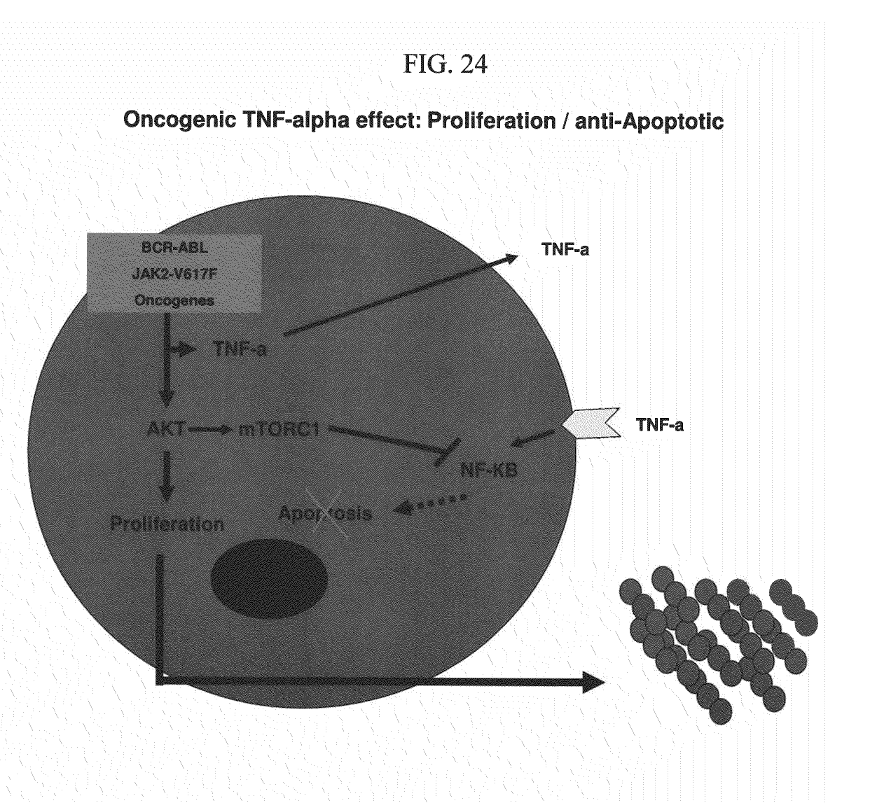

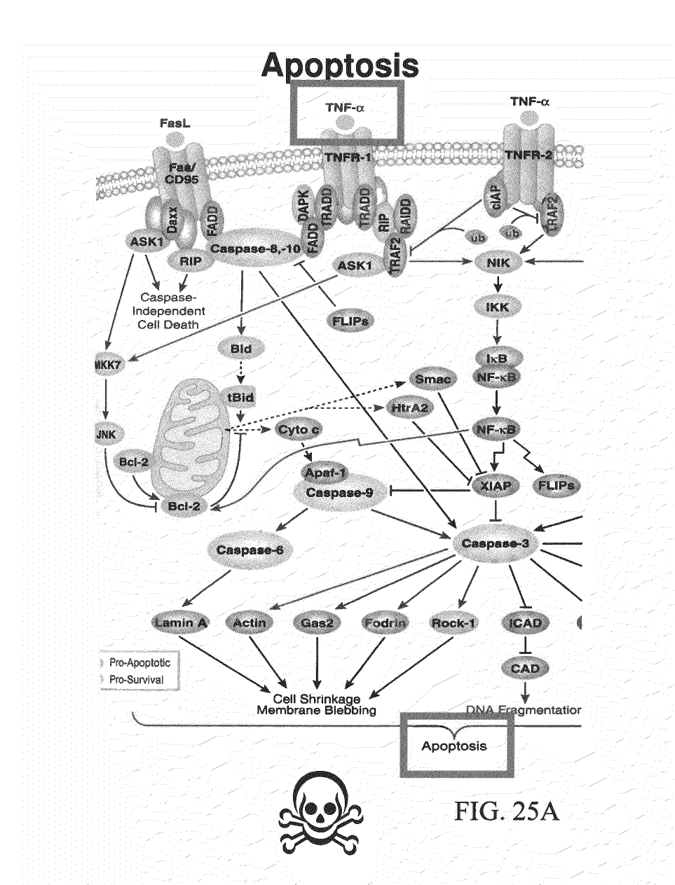

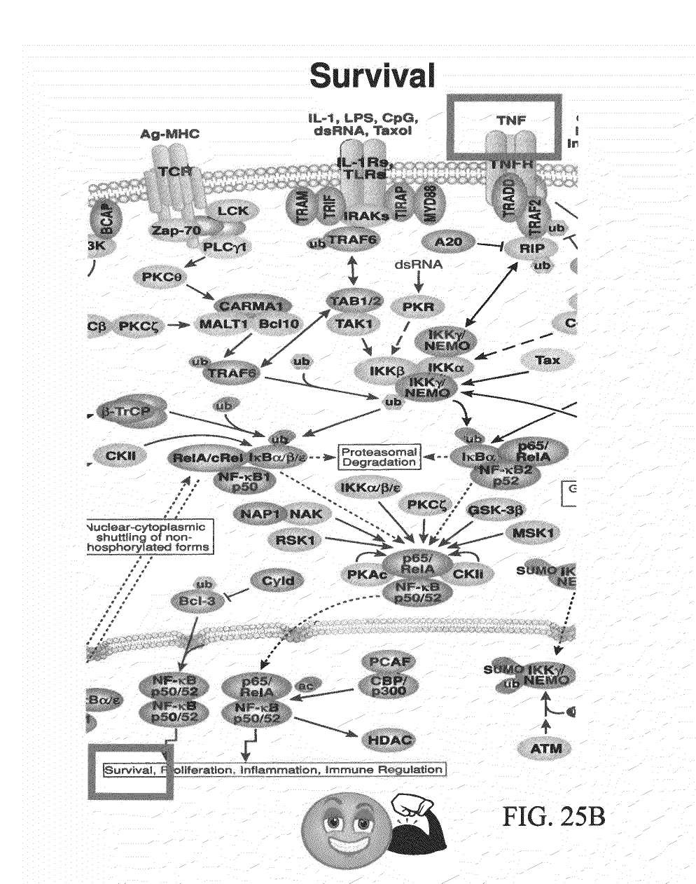

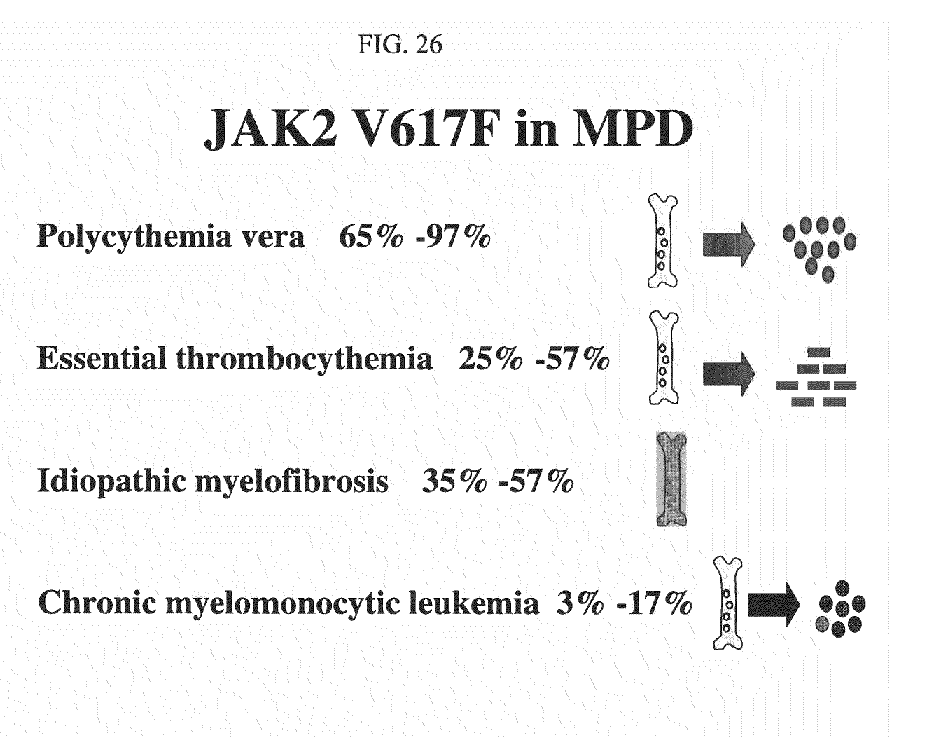

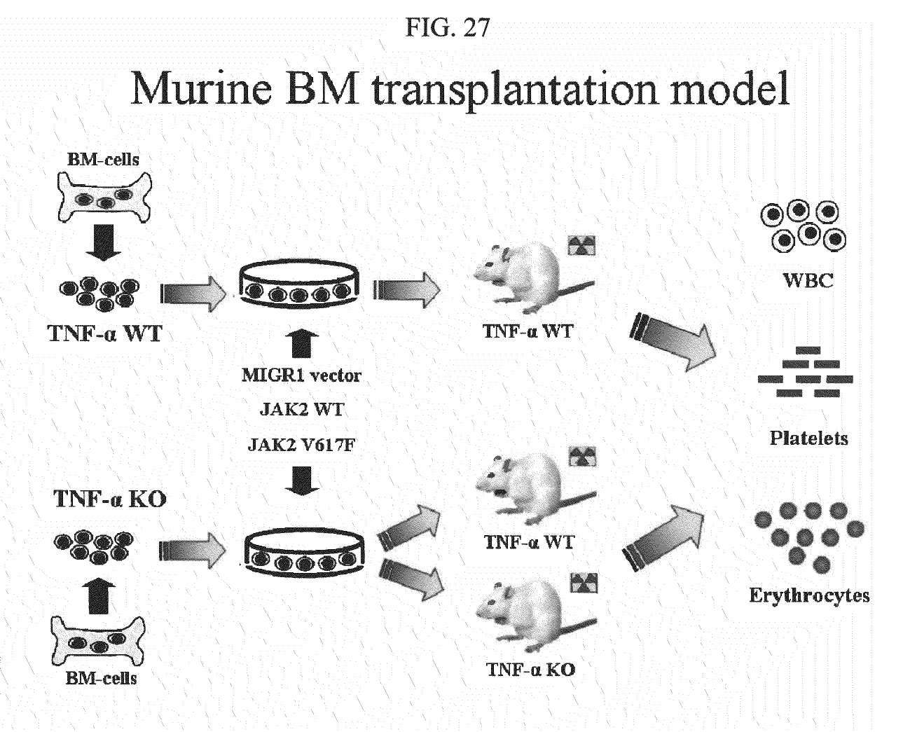

The bone marrow of mice transplanted with JAK2-V617F infected TNF-alpha wild type bone marrow showed hypercellular marrow with megakaryocytic hyperplasia and reduced erythroid cells (see FIG. 13). It also showed severe reticulin fibrosis (see FIG. 14). In contrast marrow of mice transplanted with JAK2-V617F infected TNF-alpha knock out bone marrow was normal with trilineage hematopoiesis, a myeloid to erythroid ratio of 1:1 to 2:1 and morphologically normal megakaryocytes (see FIG. 15). Reticulin fibrosis was complete absent (see FIG. 16). The spleen of mice transplanted with JAK2-V617F infected TNF-alpha wild type bone marrow showed lymphoid hyperplasia and extramedullary hematopoiesis. The red pulp was expanded, with extensive extramedullary hematopoiesis and effacement of splenic architecture (see FIG. 17). In contrast spleen of mice transplanted with JAK2-V617F infected TNF-alpha knock out bone marrow showed normal white and red pulp with minimal extramedullary hematopoiesis (see FIG. 18). This result indicates that blocking TNF-alpha is a viable treatment for JAK2-V617F induced myeloproliferative diseases, for example in conjunction with a JAK2 inhibitor.

### Example 10

### Mouse Model of Treatment with a JAK2 Kinase Inhibitor and an Inhibitor of TNF-Alpha

This example describes exemplary procedures for testing the efficay of treatment with anti JAK2 kinase inhibitor and an inhibitor of TNF-alpha, using the mouse model of example 9.

Briefly, 5-FU stimulated TNF-alpha wild type bone marrow harvested form wild type mice (normal bone marrow, able to produce TNF-alpha), is retroviral infected for three days in culture with MSCV-GFP-empty JAK2-V617F, is transplanted into lethal radiated TNF-alpha wild type mice. (empty vector and JAK2-WT is used as a control).

The mice are administered a therapeutic amount of a TNF-alpha inhibitor and JAK2 inhibitor. The TNF-alpha inhibitor can be administered at doses of 0.0001 μg/kg body weight to about 10 mg/kg body weight per dose, such as 0.0001 μg/kg body weight −0.001 μg/kg body weight per dose, 0.001 μg/kg body weight −0.01 μg/kg body weight per dose, 0.01 μg/kg body weight −0.1 μg/kg body weight per dose, 0.1 μg/kg body weight −10 μg/kg body weight per dose, 1 μg/kg body weight −100 μg/kg body weight per dose, 100 μg/kg body weight −500 μg/kg body weight per dose, 500 μg/kg body weight per dose −1000 μg/kg body weight per dose, 1.0 mg/kg body weight per dose −10 mg/kg body weight per dose or even greater. However, the particular dose can be determined by a skilled clinician. The TNF-alpha inhibitor can be administered in several doses, for example continuously, daily, weekly, or monthly.

The mice are also administered a JAK2 inhibitor. The JAK2 inhibitor can be administered at doses of 0.0001 μg/kg body weight to about 10 mg/kg body weight per dose, such as 0.0001 μg/kg body weight −0.001 μg/kg body weight per dose, 0.001 μg/kg body weight −0.01 μg/kg body weight per dose, 0.01 μg/kg body weight −0.1 μg/kg body weight per dose, 0.1 mg/kg body weight −10 μg/kg body weight per dose, 1 μg/kg body weight −100 μg/kg body weight per dose, 100 μg/kg body weight −500 μg/kg body weight per dose, 500 μg/kg body weight perdose −1000 μg/kg body weight per dose, or 1.0 mg/kg body weight per dose −10 mg/kg body weight per dose or even greater. However, the particular dose can be determined by a skilled clinician. The JAK2 inhibitor can be administered in several doses, for example continuously, daily, weekly, or monthly.

The JAK2 inhibitor can be administered concurrently or sequentially with the inhibitor of TNF-alpha. When administered sequentially the time separating the administration of the JAK2 inhibitor and the inhibitor of TNF-alpha, can be seconds, minutes, hours, days, or even weeks.

The mode of administration can be any used in the art. The amount of agent administered to the subject can be determined by a clinician, and may depend on the particular subject treated. Specific exemplary amounts are provided herein (but the disclosure is not limited to such doses).

### Example 11

### Treatment of Subjects

This example describes methods that can be used to treat a subject having a particular disease or condition, such as myeloproliferative disease that can be treated by the combination of a JAK2 kinase inhibitor and an inhibitor of TNF-alpha, for example a subject with an activating mutation in JAK2, such as the JAK2-V617F mutation, by of a combination of a JAK2 inhibitor and a TNF-alpha inhibitor. Such a therapy can be used alone, or in combination with other therapies (such as the administration of a chemotherapeutic agent).

In particular examples, the method includes screening a subject having or thought to have a particular disease or condition treatable by the combination of JAK2 kinase inhibitor and an inhibitor of TNF-alpha to identify those subjects that can benefit from administration of the JAK2 kinase inhibitor and an inhibitor of TNF-alpha. Subjects of an unknown disease status or condition can be examined to determine if they have disease or condition treatable by a combination of a JAK2 kinase inhibitor and an inhibitor of TNF-alpha, for example by determining if the subject has an activating mutation in the JAK2 kinase, such a such as the JAK2-V617F mutation. Subjects found to (or known to) have an activating mutation of JAK2, and thereby treatable by internalization of the target receptor are selected to receive a combination of a JAK2 kinase inhibitor and an inhibitor of TNF-alpha.

The subject can be administered a therapeutic amount of a JAK2 kinase inhibitor and an inhibitor of TNF-alpha. The JAK2 kinase inhibitor and the inhibitor of TNF-alpha can be administered at doses of 0.0001 μg/kg body weight to about 10 mg/kg body weight per dose, such as 0.0001 μg/kg body weight −0.001 μg/kg body weight per dose, 0.001 μg/kg body weight −0.01 μg/kg body weight per dose, 0.01 μg/kg body weight −0.1 μg/kg body weight per dose, 0.1 μg/kg body weight −10 μg/kg body weight per dose, 1 μg/kg body weight −100 μg/kg body weight per dose, 100 μg/kg body weight −500 μg/kg body weight per dose, 500 μg/kg body weight perdose −1000 μg/kg body weight per dose, or 1.0 mg/kg body weight per dose −10 mg/kg body weight per dose. However, the particular dose can be determined by a skilled clinician. The JAK2 kinase inhibitor and the inhibitor of TNF-alpha can be administered in several doses, for example continuously, daily, weekly, or monthly. The administration can concurrent or sequential.

The mode of administration can be any used in the art. The amount of the JAK2 kinase inhibitor and the inhibitor of TNF-alpha administered to the subject can be determined by a clinician, and may depend on the particular subject treated. Specific exemplary amounts are provided herein (but the disclosure is not limited to such doses).

### Example 12

### Amino Acid and Nucleotide Sequence of JAK2 and TNF-Alpha

SEQ ID NO: 1 is an exemplary amino acid sequence of wild type mouse JAK2 kinase, available at GENBANK® ACCESSION NO. NP_{—}032439, Nov. 30, 2007, which is incorporated herein be reference in its entirety.

SEQ ID NO: 2 is an exemplary nucleic acid sequence of wild type mouse JAK2 kinase, available at GENBANK® ACCESSION NO. NM_{—}008413, Nov. 30, 2007, which is incorporated herein be reference in its entirety.

SEQ ID NO: 3 is an exemplary amino acid sequence of wild type human JAK2 kinase, available at GENBANK® ACCESSION NO. NP_{—}004963, Nov. 30, 2007, which is incorporated herein be reference in its entirety.

SEQ ID NO: 4 is an exemplary nucleic acid sequence of wild type human JAK2 kinase, available at GENBANK ACCESSION NO. NM_{—}004972, Nov. 30, 2007, which is incorporated herein be reference in its entirety.

SEQ ID NO: 5 is an exemplary amino acid sequence of wild type mouse TNF-alpha, available at GENBANK ACCESSION NO. NP_{—}038721, Nov. 30, 2007, which is incorporated herein be reference in its entirety.

SEQ ID NO: 6 is an exemplary nucleic acid sequence of wild type mouse TNF-alpha, available at GENBANK ACCESSION NO. NM_{—}013693., Nov. 30, 2007, which is incorporated herein be reference in its entirety.

SEQ ID NO: 7 is an exemplary amino acid sequence of wild type human TNF-alpha, available at GENBANK ACCESSION NO. NP_{—}000585, Nov. 30, 2007, which is incorporated herein be reference in its entirety.

SEQ ID NO: 8 is an exemplary nucleic acid sequence of wild type human TNF-alpha, available at GENBANK ACCESSION NO. NM_{—}000594, Nov. 30, 2007, which is incorporated herein be reference in its entirety.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, chemical moieties, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

1. A method for treating or inhibiting a cellular proliferative disorder in a subject with an activating mutation in a Janus Activated Kinase-2 (JAK2) kinase protein, comprising:
selecting a subject with a cellular proliferative disorder having an activating mutation in a JAK2 kinase protein;
administering to the subject a therapeutically effective amount of an inhibitor of JAK2 kinase activity; and
administering to the subject a therapeutically effective amount of an inhibitor of Tumor Necrosis Factor-alpha (TNF-alpha) activity, thereby treating or inhibiting the cellular proliferative disorder in the subject.

2. The method of claim 1, wherein the cellular proliferative disorder is a myeloproliferative disorder.

3. The method of claim 2, wherein the myeloproliferative disorder is one or more of polycythemia vera (PV), essential thrombocythemia (ET), idiopathic myelofibrosis (IMF), hypereosinophilic syndrome (LIES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), unclassified myeloproliferative disease (UMPD or MPD-NC), or chronic myelogenous leukemia (CML), and AML.

4. The method of claim 3, wherein the myeloprolierative disorder is polycythemia vera (PV).

5. (canceled)

6. The method of claim 3, wherein the AML is AML-FAB M7.

7. The method of claim 1, wherein the inhibitor of TNF-alpha activity is one or more of a small molecule, an inhibitory RNA, or an antibody that specifically binds TNF-alpha.

8. (canceled)

9. The method of claim 7, wherein the antibody is one or more of Adalimumab, Etanercept, Certolizumab pegol, Golimumab, Nerelimomab, or Infliximab.

10. (canceled)

11. The method of claim 7, wherein the small molecule is LMP-160 or LMP-420.

12. The method of claim 1, wherein the inhibitor of JAK2 kinase activity is one or more of a small molecule, an inhibitory RNA, or an antibody that specifically binds JAK2.

13. (canceled)

14. The method of claim 12, wherein the small molecule is one or more of Lestaurtinb, TG101348, CYT387, AZ960, SGI-1252, WP1066, AG490, INCB18424, or SB1518.

15. The method of claim 1, wherein selecting a subject comprises detecting an activating mutation in the JAK2 kinase protein in the subject.

16. The method of claim 15, wherein the activating mutation in the JAK2 protein is a valine to phenylalanine mutation at position 617 of the JAK2 protein.

17. The method of claim 16, wherein the activating mutation in the JAK2 protein is a valine to phenylalanine mutation corresponding to position 617 of the amino acid sequence set forth as SEQ ID NO: 3 or 1.

18. The method of claim 15, wherein detecting an activating mutation in the JAK2 protein comprises, detecting a mutation in the nucleic acid sequence encoding the JAK2 protein.

19. (canceled)

20. A pharmaceutical composition comprising a therapeutically effective amount of a JAK2 inhibitor and a therapeutically effective amount of a TNF-alpha inhibitor.

21. A method for selecting a subject with a cellular proliferative disorder for treatment with an agent that inhibits (TNF)-alpha activity, comprising:
detecting the presence of an activating mutation in a JAK2 kinase protein in a biological sample obtained from the subject, wherein the presence of the activating mutation in JAK2 indicates that the subject would benefit from treatment with the agent that inhibits TNF-alpha,
thereby selecting the subject with a cellular proliferative disorder for treatment with an agent that inhibits (TNF)-alpha activity.

22. The method of claim 21, wherein the cellular proliferative disorder is a myeloproliferative disorder.

23. The method of claim 22, wherein the myeloproliferative disorder is one or more of polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), unclassified myeloproliferative disease (UMPD or MPD-NC), chronic myelogenous leukemia (CML) or acute myelogenous leukemia (AML).

24. The method of claim 23, wherein the myeloprolierative disorder is polycythemia vera (PV).

25. (canceled)

26. The method of claim 23, wherein the AML is AML-FAB M7.

27. The method of claim 21, wherein detecting the activating mutation in the JAK2 protein comprises detecting a valine to phenylalanine mutation at position 617 of the JAK2 protein.

28. The method of claim 27, wherein the activating mutation in the JAK2 protein is a valine to phenylalanine mutation corresponding to position 617 of the amino acid sequence set forth as SEQ ID NO: 3 or 1.

29. The method of claim 21, wherein detecting an activating mutation in the JAK2 protein comprises, detecting a mutation in the nucleic acid sequence encoding the JAK2 protein.

30. The method of claim 21, further comprising administering to the subject an inhibitor of TNF-alpha activity.

31. The method of claim 30, wherein the inhibitor of TNF-alpha activity is one or more of a small molecule, an inhibitory RNA, or an antibody that specifically binds TNF-alpha.

32. (canceled)

33. The method of claim 31, wherein the antibody is one or more of Adalimumab, Etanercept, Certolizumab pegol, Golimumab, Nerelimomab, or Infliximab.

34. (canceled)

35. The method of claim 31, wherein the small molecule is LMP-160 or LMP-420.

36. The method of claim 21, further comprising administering to the subject an inhibitor of JAK2 kinase activity.

37. The method of claim 36, wherein the inhibitor of JAK2 kinase activity is one or more of a small molecule, an inhibitory RNA, or an antibody that specifically binds JAK2.

38. (canceled)

39. The method of claim 37, wherein the small molecule is one or more of Lestaurtinb, TG101348, CYT387, AZ960, SGI-1252, WP1066, AG490, INCB 18424, or SB 1518.

40. A method for identifying an agent of use in treating a subject with a cellular proliferative disorder or with a predisposition for cellular proliferative disorder, comprising;
contacting an isolated cell expressing an activating mutation in the JAK2 protein with a test agent;
detecting an amount of TNF-alpha produced by the cell; and
comparing the amount of TNF-alpha produced by the cell to a control, wherein a reduction in the amount of TNF-alpha produced by the cell relative to the control indicates that the agent as useful for the treatment of a subject with the cellular proliferative disorder or with a predisposition for developing the cellular proliferative disorder.

41. The method of claim 40, wherein the cellular proliferative disorder is a myeloproliferative disorder.

42. The method of claim 41, wherein the myeloproliferative disorder is one or more of polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), unclassified myeloproliferative disease (UMPD or MPD-NC), chronic myelogenous leukemia (CML), or acute myelogenous leukemia (AML).

43. The method of claim 42, wherein the myeloprolierative disorder is polycythemia vera (PV).

44. (canceled)

45. The method of claim 41, wherein the AML is AML-FAB M7.

46. The method of claim 40, wherein the activating mutation in the JAK2 protein is a valine to phenylalanine mutation corresponding to position 617 of the amino acid sequence set forth as SEQ ID NO: 3 or 1.

47. The method of claim 40, wherein the control is a standard value or the amount of TNF-alpha produced by an isolated cell expressing the activating mutation in the JAK2 protein not contacted with the agent.

48. The method of claim 40, wherein detecting expression of TNF-alpha comprises detecting the amount of an mRNA encoding TNF-alpha.

49. The method of claim 40, wherein detecting expression of TNF-alpha comprises detecting the amount of TNF-alpha protein.
